(19) United States
(12) Patent Application Publication
(10) Pub. No.: US 2023/0131780 A1
(43) Pub. Date: Apr. 27, 2023

(54) METHODS OF DETECTING ANTIBODIES TO SARS-COV-2

(71) Applicant: PROCISEDX INC., San Diego, CA (US)

(72) Inventors: LARRY MIMMS, San Diego, CA (US); Michael Hale, San Diego, CA (US); Stefan Westin, San Diego, CA (US)

(73) Assignee: PROCISEDX INC., San Diego, CA (US)

(21) Appl. No.: 17/970,814

(22) Filed: Oct. 21, 2022

Related U.S. Application Data
(63) Continuation of application No. PCT/US2021/029839, filed on Apr. 29, 2021.
(60) Provisional application No. 63/018,121, filed on Apr. 30, 2020.

(51) Int. Cl. G01N 33/569

(52) U.S. Cl. CPC G01N 33/56983; G01N 2469/20; G01N 2333/165

(57) ABSTRACT

The present disclosure provides solution based assays for detecting antibodies induced by SARS-CoV-2 (anti-SARS-CoV-2) in a biological sample.

### CROSS-REFERENCE TO RELATED APPLICATIONS

The present application is a continuation of PCT/US2021/029839, filed Apr. 29, 2021, which application claims priority to U.S. Provisional Application No. 63/018,121, filed Apr. 30, 2020, the contents both of which are hereby incorporated by reference in their entirety for all purposes.

### BACKGROUND

The novel coronavirus disease (COVID-19) is quickly spreading across the US and deaths have been reported in all 50 states. Confirmed COVID-19 cases reported the common clinical symptoms include fever, cough, myalgia or fatigue. These symptoms are not unique features of COVID-19 because these symptoms are similar to that of other virus infected diseases such as influenza. Currently, virus nucleic acid Real Time-PCR (RT-PCR), CT imaging and some hematology parameters are the primary tools for clinical diagnosis of the infection. The virus nucleic acid RT-PCR test has become the current standard method for diagnosis of COVID-19. These real-time PCR test kits suffer from several limitations including: 1) these tests have long turnaround times and are complicated in operation; they generally take on average hours to generate results; 2) the PCR tests require certified laboratories, expensive equipment and trained technicians to operate; and 3) a relatively high false negative rate has been reported for RT-PCR of se limitations SARS-CoV-2. These limitations make RT-PCR unsuitable for use in the field for rapid and simple diagnosis and screening of patients thereby limiting the outbreak containment effort. A rapid, simple to use, sensitive, and accurate test to quickly identify infected patients of SARS-CoV-2 would be useful to prevent virus transmission and contact tracing.

It is widely accepted that IgM provides the first line of defense during viral infections, prior to the generation of adaptive, high affinity IgG responses that are important for long term immunity and immunological memory. It has been reported that after SARS infection, IgM antibody could be detected in patient blood within 3-6 days and IgG could be detected after 8 days. Since SARS-CoV-2 belongs to the same large family of viruses as those that caused the MERS and SARS outbreak, its antibody generation is similar, and detection of the IgG and IgM antibody against SARS-CoV-2 will be an indication of infection. Furthermore, detection of IgM antibodies tends to indicate a recent exposure to SARS-CoV-2, whereas detection of SARS-CoV-2 IgG antibodies may indicate more remote virus exposure. The rapid detection of both IgM and IgG antibodies will add value to the diagnosis and treatment of COVID-19 disease.

A recent study indicated that a rapid, lateral flow assay for detection of spike protein IgG and IgM demonstrated 90% sensitivity and specificity versus rtPCR.

In view of the foregoing, there is a critical need for a rapid, simple to use, sensitive, and an accurate test to quickly identify infected patients of SARS-CoV-2 to prevent virus transmission and to assure timely treatment of patients. A rapid, simple, highly sensitive test for present or remote COVID-19 infection of specific antibodies of SARS-CoV-2 in patient blood is needed. The current disclosure provides this and other needs.

### BRIEF SUMMARY

As such, in one embodiment, the present disclosure a solution phase bridging assay for detecting antibodies induced by SARS-CoV-2 (anti-SARS-CoV-2) in a biological sample from a subject, the assay comprising:
contacting the sample with a first labeled protein with a donor fluorophore;
contacting the sample with a second labeled protein with an acceptor fluorophore, wherein the first and second proteins are both spike proteins (S-protein), the first and second proteins are both nucleocapsid proteins (N-proteins), or in an alternative embodiment, two S-proteins and two N-proteins;
incubating the sample for a time sufficient to generate a ternary complex of the first labeled protein with a donor fluorophore, the second labeled protein labeled with an acceptor fluorophore and the anti-SARS-CoV-2, or in the alternative embodiment, incubating the sample for a time sufficient to generate two ternary complexes, wherein (i) the first ternary complex is a S-protein labeled with a donor fluorophore, a S-protein labeled with an acceptor fluorophore and the anti-SARS-CoV-2, the (ii) second ternary complex is a N-protein labeled with a donor fluorophore, a N-protein labeled with a different acceptor fluorophore and the anti-SARS-CoV-2; and
exciting the sample having the ternary complex(es) using a light source to detect a fluorescence emission signal associated with fluorescence resonance energy transfer (FRET) when the donor fluorophore is excited.

In another embodiment, the present disclosure provides a competitive assay method for detecting antibodies induced by SARS-CoV-2 (anti-SARS-CoV-2) in a biological sample from a subject, comprising:
contacting the sample with a complex comprising an anti-SARS-CoV-2 antibody labeled with a first fluorophore and an isolated labeled protein(s) with a second fluorophore, wherein the isolated labeled protein is a spike protein (S-protein) specific to the anti-SARS-CoV-2 antibody or a nucleocapsid proteins (N-protein) specific to the anti-SARS-CoV-2 antibody, wherein the complex emits a fluorescence emission signal associated with fluorescence resonance energy transfer (FRET) when the first fluorophore is excited using a light source;
incubating the biological sample with the complex for a time sufficient for the anti-SARS-CoV-2 in the sample to compete for binding with the anti-SARS-CoV-2 antibody labeled with the first fluorophore; and
exciting the sample using a light source to detect the fluorescence emission signal associated with FRET, wherein an absence of the fluorescence emission signal or a decrease in the fluorescence emission signal relative to the fluorescence emission signal initially emitted by the complex indicates the presence or amount of antibodies induced by SARS-CoV-2 (anti-SARS-CoV-2) in the sample.

In yet another embodiment, the present disclosure provides a sandwich assay for detecting human IgM antibodies against SARS CoV-2 protein, the method comprising:
contacting a sample with a anti-human IgM (e.g. goat, rabbit, murine, etc) labeled with a first fluorophore (e.g., a donor);
contacting the sample with a SARS CoV-2 protein labeled with a second fluorophore (e.g., an acceptor);
incubating the sample for a time sufficient to form a ternary complex comprising an anti-human IgM labeled with a first fluorophore, a SARS CoV-2 protein labeled with a second fluorophore and a human IgM antibody; and
exciting the sample having the ternary complex using a light source to detect a fluorescence emission signal associated with fluorescence resonance energy transfer (FRET).

In certain aspects, the first fluorophore is a donor fluorophore.

In certain aspects, the second fluorophore is an acceptor fluorophore.

In still yet another embodiment, the present disclosure provides a method for detecting total amount of antibody including IgG and IgM in a sample of a subject, the method comprising:
contacting a sample with (i) a first ternary complex comprising a first protein having a first fluorophore, a second protein having a second fluorophore, an anti-SARS CoV-2 IgG antibody; and (ii) a second ternary complex comprising the first protein having the first fluorophore, the second protein having the second fluorophore, an anti-SARS CoV-2 IgM antibody;
incubating the biological sample with the ternary complexes (i) and (ii) for a time sufficient for the anti-SARS-CoV-2 IgG and IgM in the sample to compete for binding for the proteins labeled with the first and the second fluorophores; and
exciting the sample using a light source to detect the fluorescence emission signal associated with FRET, wherein an absence of the fluorescence emission signal or a decrease in the fluorescence emission signal relative to the fluorescence emission signal initially emitted by the complex indicates the presence or amount of antibodies induced by SARS-CoV-2 (anti-SARS-CoV-2) in the sample.

In certain aspects, the first fluorophore is a donor fluorophore.

In certain aspects, the second fluorophore is an acceptor fluorophore.

In certain aspects, the method further comprises adding an anti-human IgM antibody having a third fluorophore to ascertain the proportion or amount of IgM which makes up the total antibodies.

In another embodiment, the present disclosure provides a multiplex inhibition assay for detecting IgG and IgM antibodies to S-protein and N-protein in a sample of a subject, the method comprising:
contacting a sample with (i) a first ternary complex comprising a S-protein having a donor fluorophore, a monoclonal anti-S-SARS CoV-2 IgG antibody having a first acceptor fluorophore attached thereto;
contacting a sample with (ii) a second ternary complex comprising a N-protein having a donor fluorophore, a monoclonal anti-N-SARS CoV-2 IgG antibody having a second acceptor fluorophore attached thereto;
incubating the biological sample with the ternary complexes (i) and (ii) for a time sufficient for the anti-SARS-CoV-2 IgG and IgM in the sample to compete for binding for the N and S labeled proteins; and
exciting the sample using a light source to detect the fluorescence emission signal associated with FRET, wherein an absence of the fluorescence emission signal or a decrease in the fluorescence emission signal relative to the fluorescence emission signal initially emitted by the complex indicates the presence or amount of IgG and IgM antibodies induced by SARS-CoV-2 (anti-SARS-CoV-2) in the sample.

In certain aspects, the S-protein is selected from the group consisting of a mammalian cell expressed recombinant spike protein of SARS-CoV-2, a fragment thereof or a synthetic S-peptide.

In certain aspects, the N-protein is selected from the group consisting of mammalian cell expressed recombinant nucleocapsid protein of SARS-CoV-2, a fragment thereof or a synthetic N-peptide.

In yet another embodiment, the present disclosure provides a kit for the detection of antibodies induced by SARS-CoV-2 (anti-SARS-CoV-2) in a biological sample from a subject, comprising:
a lyophilized first labeled protein with a donor fluorophore; and
a lyophilized second labeled protein with an acceptor fluorophore, wherein the first and second proteins are both spike proteins (S-protein) or wherein the first and second proteins are both nucleocapsid proteins (N-proteins).

These and other objects, aspects and embodiments will become more apparent when read with the accompanying detailed description and figures that follow.

### BRIEF DESCRIPTION OF THE DRAWINGS

FIG. 1 shows an embodiment of a bridging assay of the present disclosure.

FIG. 2 shows a schematic of the spike protein of SARS-CoV-2.

FIG. 3 shows an embodiment of a competitive assay of the present disclosure.

FIG. 4 shows an embodiment of detecting IgM antibodies against SARS CoV-2 protein in a sandwich format

FIG. 5 shows an embodiment of S protein Bridge assay format for total Antibody against SARS CoV-2 and multiplexed with IgM specific to anti-SARS CoV-2 IgM.

FIG. 6 shows an embodiment of a multiplex inhibition assay for detecting IgG and IgM antibodies to S-protein and N-protein.

FIG. 7 shows the structure of a cryptate donor of the present disclosure.

FIG. 8 shows the structure of an acceptor, Alexa Fluor 647, of the present disclosure.

FIG. 9 shows the distinct peaks, at about 490 nm, about 545 nm, about 580 nm, and about 620 nm, which can be used for energy transfer of a cryptate donor.

FIG. 10 shows a sample map and results of an ELISA using an N peptide as a substrate.

### DETAILED DESCRIPTION

### I. Definitions

The terms “a,” “an,” or “the” as used herein not only includes aspects with one member, but also includes aspects with more than one member.

The term “about” as used herein to modify a numerical value indicates a defined range around that value. If “X” were the value, “about X” would indicate a value from 0.9× to 1.1×, and more preferably, a value from 0.95× to 1.05×. Any reference to “about X” specifically indicates at least the values X, 0.95×, 0.96×, 0.97×, 0.98×, 0.99×, 1.01×, 1.02×, 1.03×, 1.04×, and 1.05×. Thus, “about X” is intended to teach and provide written description support for a claim limitation of, e.g., “0.98×.”

When the modifier “about” is applied to describe the beginning of a numerical range, it applies to both ends of the range. Thus, “from about 500 to 850 nm” is equivalent to “from about 500 nm to about 850 nm.” When “about” is applied to describe the first value of a set of values, it applies to all values in that set. Thus, “about 580, 700, or 850 nm” is equivalent to “about 580 nm, about 700 nm, or about 850 nm.”

“Activated acyl” as used herein includes a —C(O)-LG group. “Leaving group” or “LG” is a group that is susceptible to displacement by a nucleophilic acyl substitution (i.e., a nucleophilic addition to the carbonyl of —C(O)-LG, followed by elimination of the leaving group). Representative leaving groups include halo, cyano, azido, carboxylic acid derivatives such as t-butylcarboxy, and carbonate derivatives such as i-BuOC(O)O—. An activated acyl group may also be an activated ester as defined herein or a carboxylic acid activated by a carbodiimide to form an anhydride (preferentially cyclic) or mixed anhydride —OC(O)R^{a} or —OC(NR^{a})NHR^{b} (preferably cyclic), wherein R^{a} and R^{b} are members independently selected from the group consisting of C₁-C₆ alkyl, C₁-C₆ perfluoroalkyl, C₁-C₆ alkoxy, cyclohexyl, 3-dimethylaminopropyl, or N-morpholinoethyl. Preferred activated acyl groups include activated esters.

“Activated ester” as used herein includes a derivative of a carboxyl group that is more susceptible to displacement by nucleophilic addition and elimination than an ethyl ester group (e.g., an NHS ester, a sulfo-NHS ester, a PAM ester, or a halophenyl ester). Representative carbonyl substituents of activated esters include succinimidyloxy (—OC₄H₄NO₂), sulfosuccinimidyloxy (—OC₄H₃NO₂SO₃H), -1-oxybenzotriazolyl (—OC₆H₄N₃); 4-sulfo-2,3,5,6-tetrafluorophenyl; or an aryloxy group that is optionally substituted one or more times by electron-withdrawing substituents such as nitro, fluoro, chloro, cyano, trifluoromethyl, or combinations thereof (e.g., pentafluorophenyloxy, or 2,3,5,6-tetrafluorophenyloxy). Preferred activated esters include succinimidyloxy, sulfosuccinimidyloxy, and 2,3,5,6-tetrafluorophenyloxy esters.

“FRET partners” refer to a pair of fluorophores consisting of a donor fluorescent compound such as cryptate and an acceptor compound such as Alexa 647, when they are in proximity to one another and when they are excited at the excitation wavelength of the donor fluorescent compound, these compounds emit a FRET signal. It is known that, in order for two fluorescent compounds to be FRET partners, the emission spectrum of the donor fluorescent compound must partially overlap the excitation spectrum of the acceptor compound. The preferred FRET-partner pairs are those for which the value R0 (Forster distance, distance at which energy transfer is 50% efficient) is greater than or equal to 30 Å.

“Fluorescence resonance energy transfer (FRET)” or “Forster resonance energy transfer (FRET)” refer to a mechanism describing energy transfer between a donor compound such as cryptate and an acceptor compound such as Alexa 647, when they are in proximity to one another and when they are excited at the excitation wavelength of the donor fluorescent compound. A donor compound, initially in its electronic excited state, may transfer energy to an acceptor fluorophore through nonradiative dipole-dipole coupling. The efficiency of this energy transfer is inversely proportional to the sixth power of the distance between donor and acceptor, making FRET extremely sensitive to small changes in distance.

“FRET signal” refers to any measurable signal representative of FRET between a donor fluorescent compound and an acceptor compound. A FRET signal can therefore be a variation in the intensity or in the lifetime of luminescence of the donor fluorescent compound or of the acceptor compound when the latter is fluorescent.

“Immunoglobulin A” (IgA, also referred to as sIgA in its secretory form) is an antibody that plays a crucial role in the immune function of mucous membranes. The amount of IgA produced in association with mucosal membranes is greater than all other types of antibody combined. IgA is the main immunoglobulin found in mucous secretions, including tears, saliva, sweat, colostrum and secretions from the genitourinary tract, gastrointestinal tract, prostate and respiratory epithelium. The high prevalence of IgA in mucosal areas is a result of a cooperation between plasma cells that produce polymeric IgA (pIgA), and mucosal epithelial cells that express an immunoglobulin receptor called the polymeric Ig receptor (pIgR). pIgA is released from the nearby activated plasma cells and binds to pIgR.

“Immunoglobulin M” (IgM) refers to one of several isotypes of immunoglobulin that are produced by vertebrates. IgM is the largest antibody, and it is the first antibody to appear in the response to initial exposure to an antigen. In the case of humans and other mammals that have been studied, the spleen, where plasmablasts responsible for antibody production reside, is the major site of specific IgM production. Demonstrating IgM antibodies in a patient's serum indicates recent infection.

“Immunoglobulin G” (IgG) refers to one of several isotypes of immunoglobulin that are produced by vertebrates. IgG represents approximately 75% of serum antibodies in humans, and thus, IgG is the most common type of antibody found in blood circulation. IgG molecules are created and released by plasma B cells. Each IgG has two antigen binding sites. IgG antibodies are generated following class switching and maturation of the antibody response, thus they participate predominantly in the secondary immune response.

“Spike-protein” or “S-protein” is a transmembrane spike (S) glycoprotein of the SARS COV-2 virus that forms homotrimers protruding from the viral surface. The S-protein comprises two functional subunits responsible for binding to the host cell receptor (S1 subunit) and fusion of the viral and cellular membranes (S2 subunit). For many CoVs, S is cleaved at the boundary between the S1 and S2 subunits, which remain non-covalently bound in the prefusion conformation. The distal S1 subunit comprises the receptor-binding domain(s) and contributes to stabilization of the prefusion state of the membrane-anchored S2 subunit that contains the fusion machinery. For all CoVs, S is further cleaved by host proteases at the so-called S2′ site located immediately upstream of the fusion peptide. This cleavage has been proposed to activate the protein for membrane fusion via extensive irreversible conformational changes (See, Alexandra C. Walls et al., Cell 180, 1-12, Mar. 19, 2020). See also, surface glycoprotein [Severe acute respiratory syndrome coronavirus 2], GenBank Accession No.: QHD43416.1. Wu et al., Nature 579 (7798), 265-269 (2020), entitled “A New Coronavirus Associated with Human Respiratory Disease in China,” PUBMED 32015508. Both references hereby incorporated by reference; ACCESSION QIV15164; Submitted (6 Apr. 2020) Division of Viral Diseases, Centers for Disease Control and Prevention, 1600 Clifton Rd NE, Atlanta, Ga., 30033, USA (1273 aa) SEQ ID NO: 1.

“Nucleocapsid-protein” or “N-protein” is a structural protein of multifunction. The N protein of CoVs forms the helical ribonucleocapsid complexes with positive strand viral genomic RNA, and interacts with viral membrane protein during virion assembly, and plays an important role in enhancing the efficiency of virus replication, transcription, and assembly. The N protein is normally highly conserved, with a molecular weight of about 50 kDa. N protein has multiple functions including formation of nucleocapsids, signal transduction virus budding, RNA replication, and mRNA transcription. Nucleocapsid phosphoprotein [Severe acute respiratory syndrome coronavirus 2]; ACCESSION QIQ50129; Submitted (24 Mar. 2020) Laboratory Medicine, University of Washington, 1100 Fairview Ave N, PO Box 19024, E5-110, Seattle, Wash. 98109, USA; SEQ ID NO:4.

### II. Embodiments

The present disclosure provides a time resolved fluorescence resonance energy (trFRET) immunoassay for the detection of antibodies against a SARS COV-2 protein. A coronavirus contains four structural proteins, including spike (S), envelope (E), membrane (M), and nucleocapsid (N) proteins. In this regard, two main proteins are useful, the spike protein and the nucleocapsid protein. The spike protein (S-protein) performs two primary tasks that aid in host infection: 1) it mediates the attachment between the virus and host cell surface receptors, and 2) it facilitates viral entry into the host cell by assisting in the fusion of the viral and host cell membranes. The nucleocapsid protein (N-protein) is a structural protein that binds to the coronavirus RNA genome, thus creating a shell (or capsid) around the enclosed nucleic acid. The N-protein also 1) interacts with the viral membrane protein during viral assembly, 2) assists in RNA synthesis and folding, 3) plays a role in virus budding, and 4) affects host cell responses, including cell cycle and translation.

Fluorescence resonance energy transfer (FRET) is a process in which a donor fluorescent molecule, in an excited state, transfers excitation energy to an acceptor fluorophore when the two are brought into close proximity (˜100 Å). Upon excitation at a characteristic wavelength the energy absorbed by the donor is transferred to the acceptor, which in turn emits light energy. In certain instances, such as in a bridging assay, the level of light emitted from the acceptor fluorophore is proportional to the degree of donor/acceptor complex formation. When the specific binding event does not occur, no additional FRET signal is present.

As such, in one embodiment, the present disclosure a solution phase bridging assay for detecting antibodies induced by SARS-CoV-2 (anti-SARS-CoV-2) in a biological sample from a subject, the assay comprising:
contacting the sample with a first labeled protein with a donor fluorophore;
contacting the sample with a second labeled protein with an acceptor fluorophore, wherein the first and second proteins are both spike proteins (S-protein), the first and second proteins are both nucleocapsid proteins (N-proteins), or in an alternative embodiment, two S-proteins and two N-proteins;
incubating the sample for a time sufficient to generate a ternary complex of the first labeled protein with a donor fluorophore, the second labeled protein labeled with an acceptor fluorophore and the anti-SARS-CoV-2, or in the alternative embodiment, incubating the sample for a time sufficient to generate two ternary complexes, wherein (i) the first ternary complex is a S-protein labeled with a donor fluorophore, a S-protein labeled with an acceptor fluorophore and the anti-SARS-CoV-2, the (ii) second ternary complex is a N-protein labeled with a donor fluorophore, a N-protein labeled with an acceptor fluorophore, which acceptor fluorophore is optionally different than the S protein acceptor and the anti-SARS-CoV-2; and
exciting the sample having the ternary complex(es) using a light source to detect a fluorescence emission signal associated with fluorescence resonance energy transfer (FRET) when the donor fluorophore is excited.

In certain aspects, the antibodies are IgA antibodies, IgM antibodies, IgG antibodies or a combination thereof such as the total amount of antibodies in the sample.

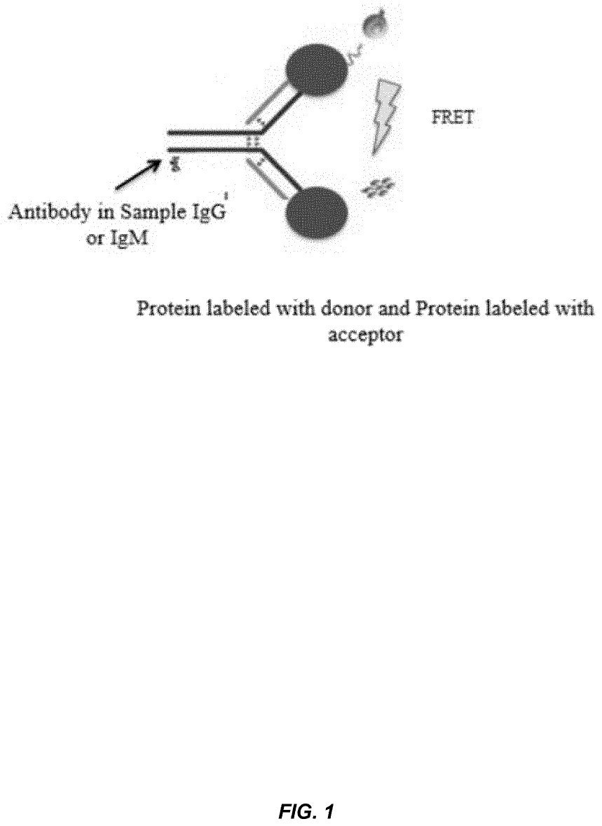

FIG. 1 shows an embodiment of a bridging assay of the present disclosure. A first SARS CoV-2 protein (e.g., spike protein, circle) is labeled with a first fluorophore (e.g., terbium cryptate, a donor) and a second SARS CoV-2 protein (e.g., spike protein, circle) is labeled with a second fluorophore (e.g., a fluorescent acceptor). When one arm of an IgG or an IgM molecule in a sample binds to the protein donor and the other arm of the same antibody binds to the protein acceptor, a trFRET signal is generated (light emission). The trFRET signal is directly proportional to the concentration of anti-SARS antibody in the sample (e.g., serum or plasma).

In certain aspects, the first and second proteins are both spike proteins (S-protein). In other aspects, the first and second proteins are both nucleocapsid proteins (N-proteins).

In an alternative embodiment, there are two S-proteins (one with a donor and the other an acceptor) and two N-proteins (one with a donor and the other an acceptor, which acceptor can optionally be a different acceptor than on the S-protein to distinguish N from S e.g., Alexa Fluor 488, Alexa Fluor 647), which generates two ternary complexes to be assayed in a multiplex fashion. Exciting the sample having the ternary complex(es) using a light source to detect a fluorescence emission signal associated with fluorescence resonance energy transfer (FRET) when the donor fluorophore is excited allows for measurement of the anti-SARS-CoV-2.

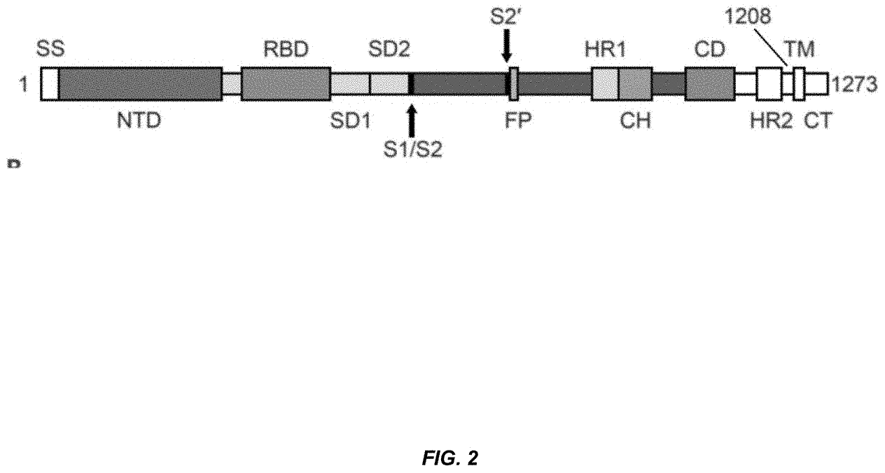

FIG. 2 is a schematic of the 2019-nCoV S-protein by domain. The following domains are shown: SS, signal sequence; S1 N terminal domain; S1 Receptor Binding Domain; S2′, S2′ protease cleavage site; FP, fusion peptide; HR1, heptad repeat 1; CH, central helix; CD, connector domain; HR2, heptad repeat 2; TM, transmembrane domain; CT, cytoplasmic tail. Arrows denote protease cleavage sites. (See, D. Wrapp et al., Science, Vol. 367, Issue 6483, pp. 1260-1263). As shown, the Spike protein (S-Protein) comprises two functional subunits responsible for binding to the host cell receptor (S1 domain) and fusion of the viral and cellular membranes (S2 domain).

In certain aspects, the S-protein is a mammalian cell expressed recombinant spike protein of SARS-CoV-2 or fragment thereof. For example, the S-protein is SEQ ID NO. 1 or a fragment thereof or synthetic peptide thereof.

In certain aspects, the S-protein is the S1 domain of the S-protein, such as the N Terminal Domain.

In certain aspects, the S-protein is the Receptor Binding Domain (RBD). The RBD is 229 amino acids, (SEQ ID NO: 2). (See, Lan, J., et al., Structure of the SARS-CoV-2 spike receptor-binding domain bound to the ACE2 receptor Nature (2020) In press, Accession: 6M0J_E.

In other aspects, the S-protein comprises a S2 portion. The S2 subunit is 132 amino acids. See, Xia, S. et al., Inhibition of SARS-CoV-2 (previously 2019-nCoV) infection by a highly potent pan-coronavirus fusion inhibitor targeting its spike protein that harbors a high capacity to mediate membrane fusion, Cell Res. 30 (4), 343-355 (2020), ACCESSION 6LXT_F (SEQ ID NO: 3).

In certain instances, each domain is targeted separately.

In other aspects, the S-protein is a synthetic protein as shown below in Table 1:

| No | Sequence |
|---|---|
| P1 | KGIYQTSNFRVQPTESIVRFPNITNLCPFGEVFNATK |
| | (SEQ ID NO: 5) |
| P2 | GAALQIPFAMQMAYRFNGIGVTQNVLYENQK |
| | (SEQ ID NO: 6) |
| P3 | AISSVLNDILSRLDKVEAEVQIDRLITGRLQSLQTK |
| | (SEQ ID NO: 7) |
| P4 | YVTQQLIRAAEIRASANLAATKMSECVLGQSKRVDK |
| | (SEQ ID NO: 8) |

In certain other aspects, synthetic peptides derived from SARS coronavirus S protein can be used (See, Wei Lu et al. FEBS Lett. 2005 Apr. 11; 579(10): 2130-2136) as the S-protein. Table 2 lists synthetic peptides based on bioinformatics analysis useful in the present invention.

| No | Position | Peptide Sequence | Putative Location |
|---|---|---|---|
| 2-P1 | 34-54 | TSSMRGVYYPDEIFRSDTLYL | S1 |
| | | (SEQ ID NO: 9) | |
| 2-P2 | 94-113 | KSNVVRGWVFGSTMNNKSQS | S1 |
| | | (SEQ ID NO: 10) | |
| 2-P3 | 197-216 | YKGYQPIDVVRDLPSGFNTL (SEQ | S1 |
| | | ID NO: 11) | |
| 2-P4 | 273-293 | TDAVDCSQNPLAELKCSVKSF | S1 |
| | | (SEQ ID NO: 12) | |
| 2-P5 | 297-316 | KGIYQTSNFRVVPSGDVVRF (SEQ | S1 |
| | | ID NO: 13) | |
| 2-P6 | 332-351 | TKFPSVYAWERKKISNCVAD | S1 |
| | | (SEQ ID NO: 14) | |
| 2-P7 | 436-455 | YNYKYRYLRHGKLRPFERDI | S1 |
| | | (SEQ ID NO: 15) | |
| 2-P8 | 540-559 | PSSKRFQPFQQFGRDVSDFT (SEQ | S1 |
| | | ID NO: 16) | |
| 2-P9 | 731-753 | CANLLLQYGSFCTQLNRALSGIA | S2 |
| | | (SEQ ID NO: 17) | |
| 2-P10 | 758-780 | RNTREVFAQVKQMYKTPTLKYFG | S2 |
| | | (SEQ ID NO: 18) | |

In certain other instances, synthetic peptides outside the spike protein heptad repeat regions as potent inhibitors of SARS-associated coronavirus can be used as the S-protein (See, Bo-Jian Zheng et al., Antiviral Therapy 10:393-403 2005). The following Table 3 lists synthetic peptides useful in the present invention.

| No | Position | Peptide Sequence | Location |
|---|---|---|---|
| 3-P1 | 220-239 | FKLPLGIN(K)ITNFRAILTAFS(L) (SEQ | S1 |
| | | ID NO: 19) | |
| 3-P2 | 259-278 | PTT(K)FMLKYDENGTITDAVDC | S1 |
| | | (SEQ ID NO: 20) | |
| 3-P3 | 354-373 | VLYNSTF(S)FSTFKCYGVSATK (SEQ | S1 |
| | | ID NO: 21) | |
| 3-P4 | 470-489 | PALNCYWPLN(K)DYGFYTTSGI | S1 |
| | | (SEQ ID NO: 22) | |
| 3-P5 | 553-572 | RDVSDF(I)TDSVRDPKTSEILD (SEQ | S1 |
| | | ID NO: 23) | |
| 3-P6 | 598-617 | YQDVNCTDVS(P)TAIHADQLTP (SEQ | S1 |
| | | ID NO: 24) | |
| 3-P7 | 690-709 | SNNTIAIPTNFS(L)ISITTEVM (SEQ ID | S1 |
| | | NO: 25) | |
| 3-P8 | 737-756 | QYGSFCT(A)QLNRALSGIAA(V)EQ | S1 |
| | | (SEQ ID NO: 26) | |
| 3-P9 | 890-909 | GIGVT(A)QNVLYENQKQIANQF | S2 |
| | | (SEQ ID NO: 27) | |
| 3-P10 | 1161-1180 | IQK(E)EIDRLNEVAKNLNESLI (SEQ | S2 |
| | | ID NO: 28) | |

The S protein of SARS-CoV consists of 1255 amino acid residues. Ten peptides of Table 3 were designed to block viral entry based on the hypothesis that the residue variations between human SARS-CoV and animal SARS-CoV-like viruses might determine the preference of viral infection between human and animals. Amino acid variation(s) in each peptide is shown in italic and the alternative amino acid(s) identified from animal SARS-CoV-like virus is shown in parentheses. Peptides with strong anti-SARS-CoV activities are 3-P2, 3-P6, 3-P8 and 3-P10.

FIG. 1 shows a ternary complex of the first labeled protein with a donor fluorophore, the second labeled protein labeled with an acceptor fluorophore and the anti-SARS-CoV-2 (e.g. IgG antibody). The labeled proteins can be S-protein or N-protein or a combination thereof.

In certain aspects, the N-protein is a mammalian cell expressed recombinant nucleocapsid protein of SARS-CoV-2 or fragment thereof. For example, the N-protein is SEQ ID NO. 4 or fragment thereof, or a synthetic peptide.

In certain aspects, the N-protein is a synthetic peptide derived from B cell epitopes from (Ahmed et al 2020), Viruses 2020, 12, 254 2020 shown in Table 4.

| No. | Peptide Sequence |
|---|---|
| 4-P1 | GRRPQGLPNNTASWFTALTQHGKEDLK (SEQ ID NO: 29) |
| 4-P2 | QLPQGTTLPKGFYAEGSRGGSQASSRSSSRSRK |
| | (SEQ ID NO: 30) |
| 4-P3 | QQQGQTVTKKSAAEASKKPRQKRTATKAYNVTQAFGRRGPEQT |
| | QGNFK (SEQ ID NO: 31) |
| 4-P4 | IRQGTDYKHWPQIAQFAPSASAFFGMSRIGMEVTPSGTWK |
| | (SEQ ID NO: 32) |
| 4-P5 | LLNKHIDAYKTEPKTFPPKDKKKKADEK |
| | (SEQ ID NO: 33) |
| 4-P6 | TQALPQRQKKQQTVTLLPAADLDDFSK (SEQ ID NO: 34) |
| 4-P7 | PSDSTGSNQNGERSGARSKQGRRPQK (SEQ ID NO: 35) |
| 4-P8 | LLNKHIDAYKTEPKTFPPKDKKKKADEK |
| | (SEQ ID NO: 36) |

In certain other aspects, synthetic peptides derived from SARS coronavirus N protein can be used as the N-Protein (See, J. Wang et al., Clinical Chemistry, Volume 49, Issue 12, 1 Dec. 2003, Pages 1989-1996). Table 5 lists synthetic peptides useful in the present invention.

| No | Position | Peptide Sequence | Location |
|---|---|---|---|
| 5-P1 | 1-23 | MSDNGPQSNQRSAPRITFGGPTD | N1 |
| | | (SEQ ID NO: 37) | |
| 5-P2 | 21-42 | PTDSTDNNQNGGRNGARPKQRR | N21 |
| | | (SEQ ID NO: 38) | |
| 5-P3 | 35-56 | GARPKQRRPQGLPNNTASWFTA | N35 |
| | | (SEQ ID NO: 39) | |
| 5-P4 | 66-87 | QLPQGTTLPKGFYAEGSRGGSQ (SEQ | N66 |
| | | ID NO: 40) | |
| 5-P5 | 99-120 | DGKMKELSPRWYFYYLGTGPEA | N99 |
| | | (SEQ ID NO: 41) | |
| 5-P6 | 177-198 | SRGGSQASSRSSSRSRGNSRNS (SEQ | N177 |
| | | ID NO: 42) | |
| 5-P7 | 196-217 | RNSTPGSSRGNSP ARMAS GGGE | N196 |
| | | (SEQ ID NO: 43) | |
| 5-P8 | 215-239 | GGETALALLLLDRLNQLESKVSGKG | N215 |
| | | (SEQ ID NO: 44) | |
| 5-P9 | 245-268 | QTVTKKSAAEASKKPRQKRTATKQ | N245 |
| | | (SEQ ID NO: 45) | |
| 5-P10 | 258-279 | KPRQKRTATKQYNVTQAFGRRG | N258 |
| | | (SEQ ID NO: 46) | |
| 5-P11 | 355-376 | NKHIDAYKTFPPTEPKKDKKKK | N355 |
| | | (SEQ ID NO: 47) | |
| 5-P12 | 371-390 | KDKKKKTDEAQPLPQRQKKQ (SEQ | N371 |
| | | ID NO: 48) | |
| 5-P13 | 385-407 | QRQKKQPTVTLLPAADMDDFSRQ | N385 |
| | | (SEQ ID NO: 49) | |
| 5-P14 | 401-422 | MDDFSRQLQNSMSGASADSTQA | N401 |
| | | (SEQ ID NO: 50) | |

In certain aspects, the FRET assay is a time-resolved FRET assay. The fluorescence emission signal or measured FRET signal is directly correlated with the biological phenomenon studied. In fact, the level of energy transfer between the donor compound and the acceptor fluorescent compound is proportional to the reciprocal of the distance between these compounds to the 6^{th} power. For the donor/acceptor pairs commonly used by those skilled in the art, the distance Ro (corresponding to a transfer efficiency of 50%) is in the order of 1, 5, 10, 20 or 30 nanometers.

In another embodiment, the present disclosure provides a competitive assay method for detecting antibodies induced by SARS-CoV-2 (anti-SARS-CoV-2) in a biological sample from a subject, comprising:
contacting the sample with a complex comprising an anti-SARS-CoV-2 antibody labeled with a first fluorophore and an isolated labeled protein(s) with a second fluorophore, wherein the isolated labeled protein is a spike protein (S-protein) specific to the anti-SARS-CoV-2 antibody or a nucleocapsid proteins (N-protein) specific to the anti-SARS-CoV-2 antibody, wherein the complex emits a fluorescence emission signal associated with fluorescence resonance energy transfer (FRET) when the first fluorophore is excited using a light source;
incubating the biological sample with the complex for a time sufficient for the anti-SARS-CoV-2 in the sample to compete for binding with the anti-SARS-CoV-2 antibody labeled with the first fluorophore; and
exciting the sample using a light source to detect the fluorescence emission signal associated with FRET, wherein an absence of the fluorescence emission signal or a decrease in the fluorescence emission signal relative to the fluorescence emission signal initially emitted by the complex indicates the presence or amount of antibodies induced by SARS-CoV-2 (anti-SARS-CoV-2) in the sample.

In certain aspects, the first fluorophore is a donor fluorophore.

In certain aspects, the second fluorophore is an acceptor fluorophore.

In certain aspects, the antibodies are IgA antibodies, IgM antibodies, IgG antibodies or a combination thereof such as the total amount of antibodies in the sample.

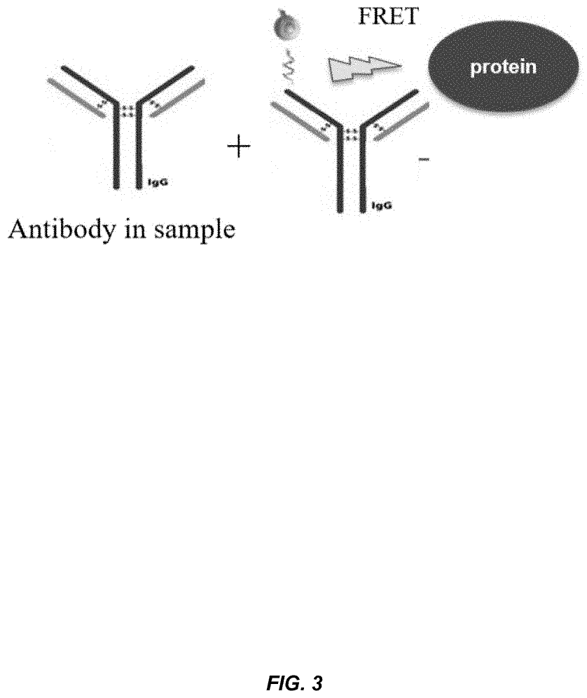

FIG. 3 shows an embodiment of a competitive assay of the present disclosure. As shown therein, a complex comprises an anti-SARS-CoV-2 antibody labeled with a first fluorophore (e.g., a donor) and an isolated labeled protein(s) with a second fluorophore (e.g., an acceptor). The isolated labeled protein can be a spike protein (S-protein) specific to an anti-SARS-CoV-2 antibody or a nucleocapsid proteins (N-protein) specific to an anti-SARS-CoV-2 antibody. A decrease in the fluorescence emission signal relative to the fluorescence emission signal initially emitted by the complex indicates the presence or amount of antibodies induced by SARS-CoV-2 (anti-SARS-CoV-2) in the sample. In other words, the presence of antibodies in the sample known to bind the protein (S-protein of N-protein) interfere with the ability of FRET to occur, thus leading to a concentration-dependent decrease of the ratio of acceptor:donor fluorescence emission. The more antibody present in the sample, the more the decrease of the ratio of acceptor:donor fluorescence emission.

During operation of the assay, the sample is incubated with the labeled antibody-protein complex for a time sufficient for the anti-SARS-CoV-2 in the sample to compete for binding with the anti-SARS-CoV-2 antibody labeled with the first fluorophore. The amount or concentration of the antibody in the sample is proportional to a concentration-dependent decrease of the ratio of fluorescence emission.

In certain aspects of the embodiments, the sample is a biological sample. Suitable biological samples include, but are not limited to, whole blood, plasma, serum, blood cells, cell samples, urine, spinal fluid, sweat, tear fluid, saliva, oral fluid, skin, mucous membrane, and hair. As a sample, whole blood, plasma, serum, blood cells, saliva and such are preferred, and whole blood, serum, plasma and saliva are particularly preferred. Whole blood includes samples of whole blood-derived blood cell fractions admixed with plasma. With regard to these samples, samples subjected to pretreatments such as hemolysis, separation, dilution, concentration, and purification can be used. In one aspect, the biological sample is a whole blood or a serum sample or a plasma sample.

In certain aspects, the antibodies being detected are IgA antibodies such as wherein the biological sample is an oral fluid or saliva.

In certain aspects, human IgM antibodies against SARS CoV-2 protein are detected in a sandwich format. For example, the complex comprises an anti-human IgM (goat, rabbit, murine, etc) labeled with a first fluorophore (e.g., a donor) and a SARS CoV-2 protein labeled with a second fluorophore (e.g., an acceptor) form a binding complex when human IgM antibody against SARS CoV-2 protein present in the sample.

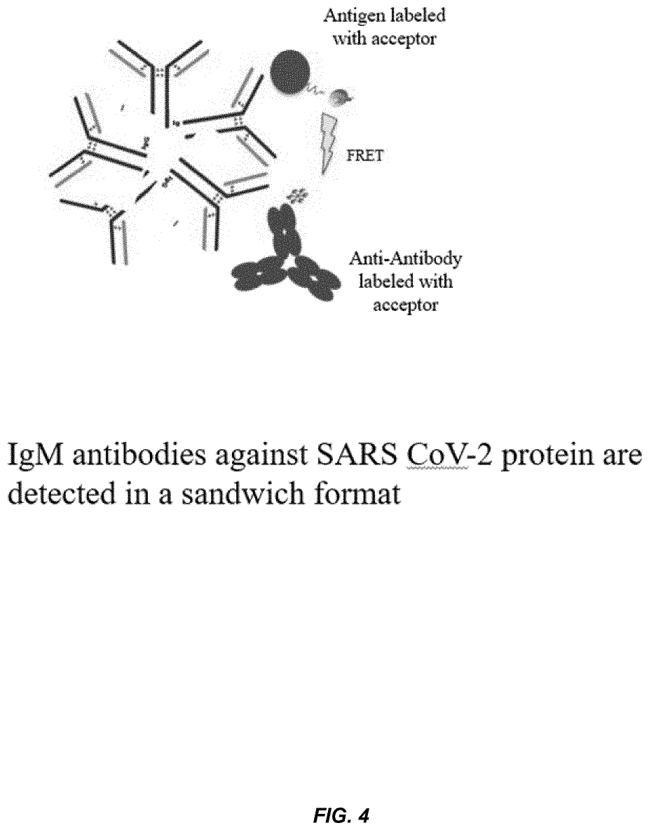

FIG. 4 shows an embodiment of a sandwich assay of the present disclosure. As shown therein, the present disclosure provides a sandwich assay for detecting human IgM antibodies against SARS CoV-2 protein. In this assay, the sample is contacted with an anti-human IgM (goat, rabbit, murine) labeled with a first fluorophore (e.g., a donor). Next, the sample is contacted with a SARS CoV-2 protein labeled with a second fluorophore (e.g., an acceptor). The sample is incubated for a time sufficient to form a ternary complex comprising an anti-human IgM labeled with a first fluorophore, a SARS CoV-2 protein labeled with a second fluorophore and a human IgM antibody; and then exciting the sample to detect a fluorescence emission signal associated with fluorescence resonance energy transfer (FRET).

In yet another embodiment, the present disclosure provides a sandwich assay for detecting human IgM antibodies against SARS CoV-2 protein, the method comprising:
contacting a sample with a goat anti-human IgM labeled with a first fluorophore (e.g., a donor);
contacting the sample with a SARS CoV-2 protein labeled with a second fluorophore (e.g., an acceptor);
incubating the sample for a time sufficient to form a ternary complex comprising a goat anti-human IgM labeled with a first fluorophore, a SARS CoV-2 protein labeled with a second fluorophore and a human IgM antibody; and
exciting the sample having the ternary complex using a light source to detect a fluorescence emission signal associated with fluorescence resonance energy transfer (FRET).

In certain aspects, the first fluorophore is a donor fluorophore.

In certain aspects, the second fluorophore is an acceptor fluorophore.

In certain aspects, the SARS CoV-2 protein is a S-protein selected from the group consisting of a mammalian cell expressed recombinant spike protein of SARS-CoV-2, a fragment thereof or a synthetic S-peptide.

In certain aspects, the SARS CoV-2 protein is a N-protein selected from the group consisting of mammalian cell expressed recombinant nucleocapsid protein of SARS-CoV-2, a fragment thereof or a synthetic N-peptide.

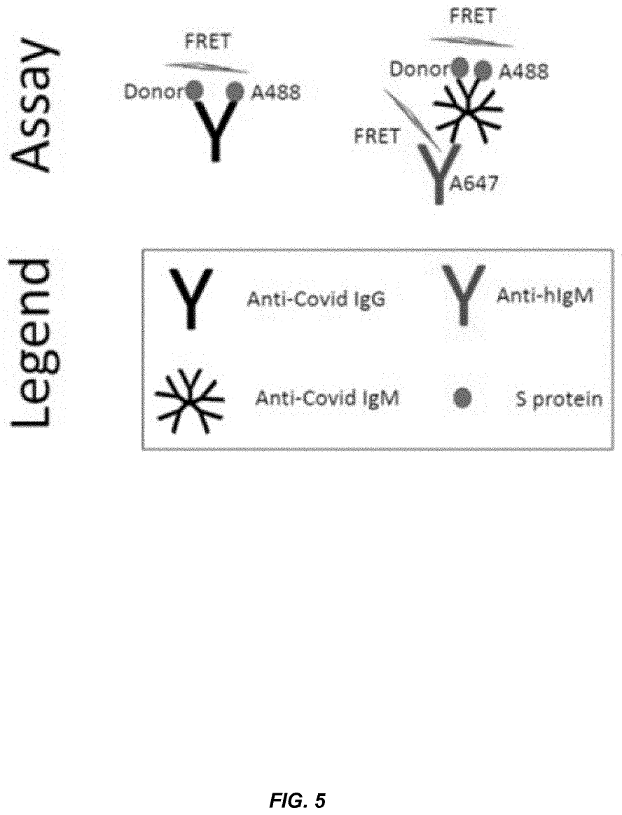

FIG. 5 shows an embodiment of the disclosure for detecting total amount of antibody including IgG and IgM in a sample of a subject. The method comprises contacting a sample with (i) a first ternary complex comprising a first protein having a first fluorophore, a second protein having a second fluorophore, and an anti-SARS CoV-2 IgG antibody. The method includes contacting the sample with (ii) a second ternary complex comprising the first protein having the first fluorophore, the second protein having the second fluorophore, and an anti-SARS CoV-2 IgM antibody. Next, the ternary complexes (i) and (ii) are incubated for a time sufficient for the anti-SARS-CoV-2 IgG and IgM in the sample to compete for binding for the proteins labeled with the first and the second fluorophores; and exciting the sample using a light source to detect the fluorescence emission signal associated with FRET. A decrease in the fluorescence emission signal relative to the fluorescence emission signal initially emitted by the complex indicates the presence or amount of antibodies induced by SARS-CoV-2 (anti-SARS-CoV-2) in the sample.

As such, in still yet another embodiment, the present disclosure provides a method for detecting total amount of antibody including IgG and IgM in a sample of a subject, the method comprising:
contacting a sample with (i) a first ternary complex comprising a first protein having a first fluorophore, a second protein having a second fluorophore, an anti-SARS CoV-2 IgG antibody; and (ii) a second ternary complex comprising the first protein having the first fluorophore, the second protein having the second fluorophore, an anti-SARS CoV-2 IgM antibody;
incubating the biological sample with the ternary complexes (i) and (ii) for a time sufficient for the anti-SARS-CoV-2 IgG and IgM in the sample to compete for binding for the proteins labeled with the first and the second fluorophores; and
exciting the sample using a light source to detect the fluorescence emission signal associated with FRET, wherein an absence of the fluorescence emission signal or a decrease in the fluorescence emission signal relative to the fluorescence emission signal initially emitted by the complex indicates the presence or amount of antibodies induced by SARS-CoV-2 (anti-SARS-CoV-2) in the sample.

In certain aspects, the method further comprises adding an anti-human IgM antibody having a third fluorophore (FIG. 5) (A647) to ascertain the proportion or amount of IgM which makes up the total antibodies.

In certain aspects, the first fluorophore is a donor fluorophore.

In certain aspects, the second fluorophore is an acceptor fluorophore.

In certain aspects, the first and second proteins are both spike proteins (S-protein). In other aspects, the first and second proteins are both nucleocapsid proteins (N-proteins).

In certain aspects, the S-protein is selected from the group consisting of a mammalian cell expressed recombinant spike protein of SARS-CoV-2, a fragment thereof or a synthetic S-peptide.

In certain aspects, the N-protein is selected from the group consisting of mammalian cell expressed recombinant nucleocapsid protein of SARS-CoV-2, a fragment thereof or a synthetic N-peptide.

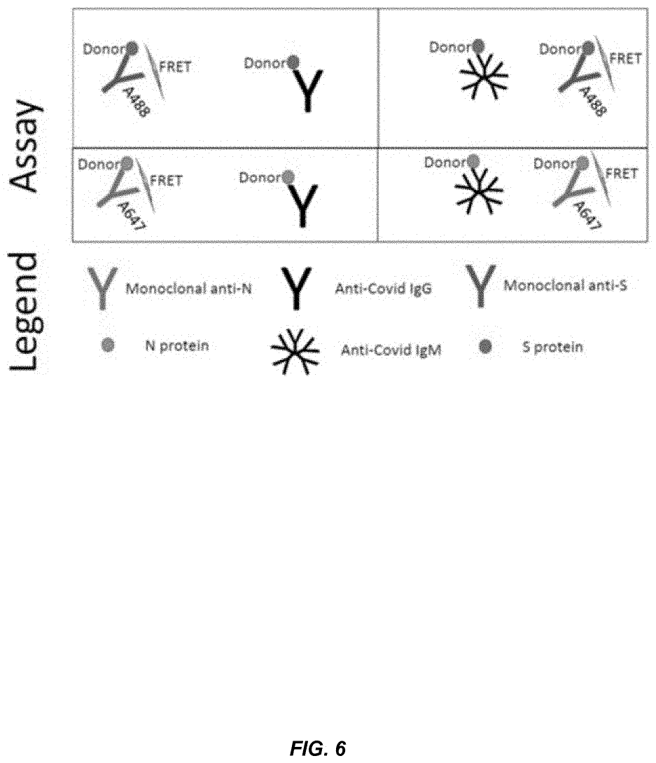

FIG. 6 shows an embodiment of the present disclosure for a multiplex inhibition assay for detecting IgG and IgM antibodies to S-protein and N-proteins. The method includes contacting a sample with (i) a first ternary complex comprising a S-protein having a donor fluorophore, a monoclonal anti-S-SARS CoV-2 IgG antibody having a first acceptor fluorophore attached thereto. In addition, contacting a sample with (ii) a second ternary complex comprising a N-protein having a donor fluorophore, a monoclonal anti-N-SARS CoV-2 IgG antibody having a second acceptor fluorophore attached thereto. The sample is incubated with the ternary complexes (i) and (ii) for a time sufficient for the anti-SARS-CoV-2 IgG and IgM in the sample to compete for binding for the N and S labeled proteins; and exciting the sample using a light source to detect the fluorescence emission signal associated with FRET, wherein an absence of the fluorescence emission signal or a decrease in the fluorescence emission signal relative to the fluorescence emission signal initially emitted by the complex indicates the presence or amount of IgG and IgM antibodies induced by SARS-CoV-2 (anti-SARS-CoV-2) in the sample.

As such, in another embodiment, the present disclosure provides a multiplex inhibition assay for detecting IgG and IgM antibodies to S-protein and N-protein in a sample of a subject, the method comprising:
contacting a sample with (i) a first ternary complex comprising a S-protein having a donor fluorophore, a monoclonal anti-S-SARS CoV-2 IgG antibody having a first acceptor fluorophore attached thereto;
contacting a sample with (ii) a second ternary complex comprising a N-protein having a donor fluorophore, a monoclonal anti-N-SARS CoV-2 IgG antibody having a second acceptor fluorophore attached thereto;
incubating the biological sample with the ternary complexes (i) and (ii) for a time sufficient for the anti-SARS-CoV-2 IgG and IgM in the sample to compete for binding for the N and S labeled proteins; and
exciting the sample using a light source to detect the fluorescence emission signal associated with FRET, wherein an absence of the fluorescence emission signal or a decrease in the fluorescence emission signal relative to the fluorescence emission signal initially emitted by the complex indicates the presence or amount of IgG and IgM antibodies induced by SARS-CoV-2 (anti-SARS-CoV-2) in the sample.

In certain aspects, the S-protein is selected from the group consisting of a mammalian cell expressed recombinant spike protein of SARS-CoV-2, a fragment thereof or a synthetic S-peptide.

In certain aspects, the N-protein is selected from the group consisting of mammalian cell expressed recombinant nucleocapsid protein of SARS-CoV-2, a fragment thereof or a synthetic N-peptide.

In certain aspects of the embodiments, the FRET energy donor compound is a cryptate, such as a lanthanide cryptate.

In certain aspects of the embodiments, the cryptate has an absorption wavelength between about 300 nm to about 400 nm such as about 325 nm to about 375 nm. In certain aspects, the cryptate dyes have four fluorescence emission peaks at about 490 nm, about 548 nm, about 587 nm, and 621 nm. Thus, as a donor, the cryptate is compatible with fluorescein-like (green zone) molecules, Cy5, DY-647-like (red zone) acceptors, Allophycocyanin (APC), or Phycoeruythrin (PE) to perform TR-FRET experiments.

In certain aspects of the embodiments, the introduction of a time delay between a flash excitation and the measurement of the fluorescence at the acceptor emission wavelength allows to discriminate long lived from short-lived fluorescence and to increase signal-to-noise ratio.

In certain aspects of the embodiments, the detection device detects time-resolved (tr) fluorescent signal from both the donor and FRET acceptor emission. Time-resolved (tr) FRET is a technique to improve signal to noise by removing short-lived fluorescent signals originating from the sample. The donor fluorophore is excited using a pulse of light. The emission from both the donor and acceptor signals are read after a time delay from the end of the excitation pulse. Noise is reduced as background fluorescence from nonspecific sources decay more rapidly than the emitted light from the donor allowing the acceptor signal to be read long after the nonspecific fluorescence has passed.

In certain aspects of the embodiments, the assay uses a donor fluorophore consisting of terbium bound within a cryptate. The terbium cryptate can be excited with a 365 nm UV LED. The terbium cryptate emits at four (4) wavelengths within the visible region. In one aspect, the assay uses the lowest donor emission energy peak of 620 nm as the donor signal within the assay. In certain aspects, the acceptor fluorophore, when in very close proximity, is excited by the highest energy terbium cryptate emission peak of 490 nm causing light emission at 520 nm. Both the 620 nm and 520 nm emission wavelengths are measured independently in a device or instrument and results can be reported as RFU ratio 620/520.

1. Cryptates as FRET Donors

In certain aspects, the terbium cryptate molecule “Lumi4-Tb” from Lumiphore, marketed by Cisbio bioassays is used as the cryptate donor. The terbium cryptate “Lumi4-Tb” having the formula below, which can be coupled to an antibody by a reactive group, in this case, for example, an NHS ester:

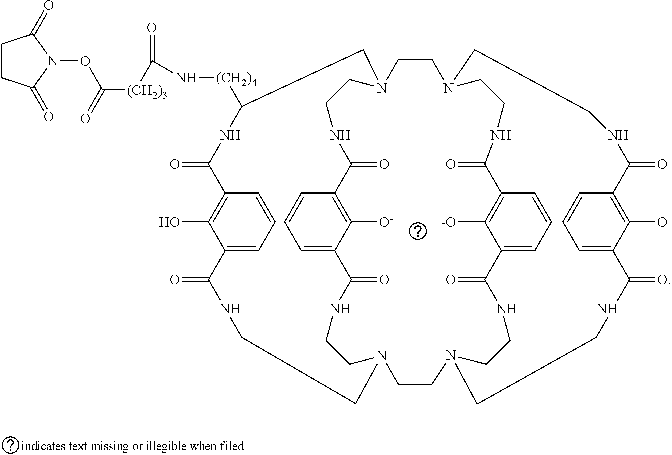

An activated ester (an NHS ester) can react with a primary amine on an antibody to make a stable amide bond. A maleimide on the cryptate and a thiol on the antibody can react together and make a thioether. Alkyl halides react with amines and thiols to make alkylamines and thioethers, respectively. Any derivative providing a reactive moiety that can be conjugated to an antibody can be utilized herein.

In certain other aspects, cryptates disclosed in WO2015157057, titled “Macrocycles” are suitable for use in the present disclosure. This publication contains cryptate molecules useful for labeling biomolecules. As disclosed therein, certain of the cryptates have the structure as follows:

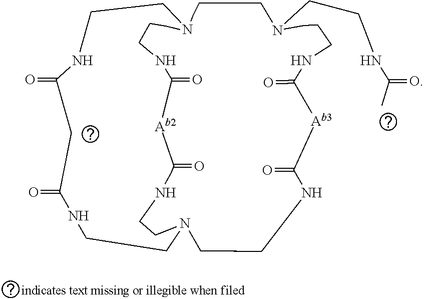

In certain other aspects, a terbium cryptate useful in the present disclosure is shown below:

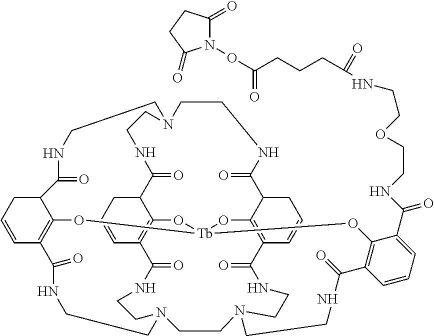

In certain aspects, the cryptates that are useful in the present invention are disclosed in WO 2018/130988, published Jul. 19, 2018. As disclosed therein, the compounds of Formula I are useful as FRET donors in the present disclosure:

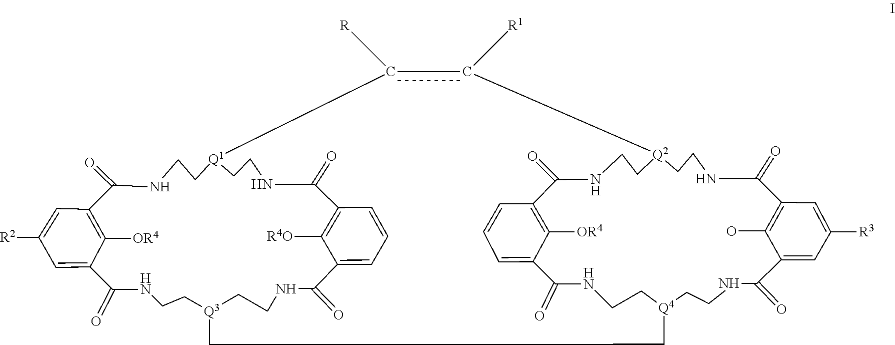

wherein when the dotted line is present, R and R¹ are each independently selected from the group consisting of hydrogen, halogen, hydroxyl, alkyl optionally substituted with one or more halogen atoms, carboxyl, alkoxycarbonyl, amido, sulfonato, alkoxycarbonylalkyl or alkylcarbonylalkoxy or alternatively, R and R¹ join to form an optionally substituted cyclopropyl group wherein the dotted bond is absent;
R² and R³ are each independently a member selected from the group consisting of hydrogen, halogen, SO₃H, —SO₂—X, wherein X is a halogen, optionally substituted alkyl, optionally substituted aryl, optionally substituted alkenyl, optionally substituted alkynyl, optionally substituted cycloalkyl, or an activated group that can be linked to a biomolecule, wherein the activated group is a member selected from the group consisting of a halogen, an activated ester, an activated acyl, optionally substituted alkylsulfonate ester, optionally substituted arylsulfonate ester, amino, formyl, glycidyl, halo, haloacetamidyl, haloalkyl, hydrazinyl, imido ester, isocyanato, isothiocyanato, maleimidyl, mercapto, alkynyl, hydroxyl, alkoxy, amino, cyano, carboxyl, alkoxycarbonyl, amido, sulfonato, alkoxycarbonylalkyl, cyclic anhydride, alkoxyalkyl, a water solubilizing group or L;
R⁴ are each independently a hydrogen, C₁-C₆ alkyl, or alternatively, 3 of the R⁴ groups are absent and the resulting oxides are chelated to a lanthanide cation; and
Q¹-Q⁴ are each independently a member selected from the group of carbon or nitrogen.

2. FRET Acceptors

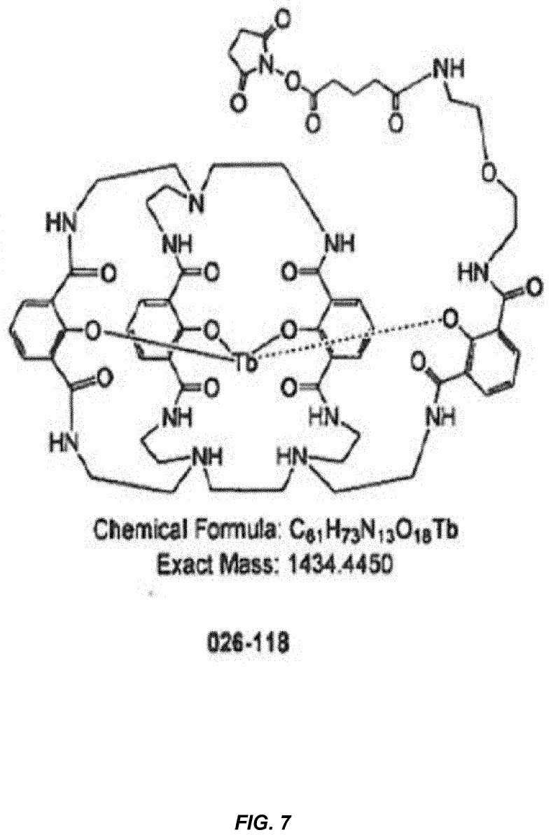

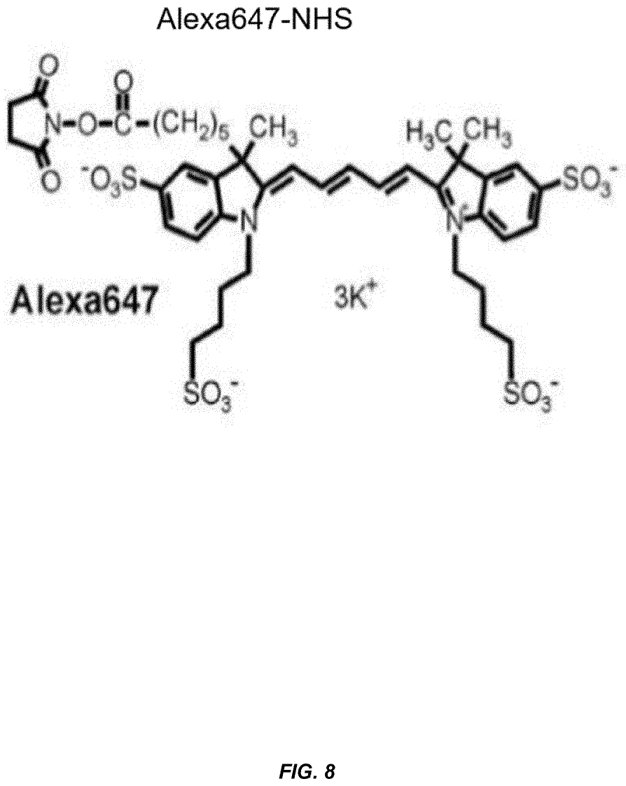

In order to detect a FRET signal, a FRET acceptor is required. The FRET acceptor has an excitation wavelength that overlaps with an emission wavelength of the FRET donor. The FRET signal of the acceptor is proportional to the concentration level of antibody present in the sample. A cryptate donor can be used to label the first protein (FIG. 7). Lumi4 has 4 spectrally distinct peaks, at about 490 nm, about 545 nm, about 580 nm, and about 620 nm, which can be used for energy transfer (FIG. 8). An acceptor can be used to label the second protein.

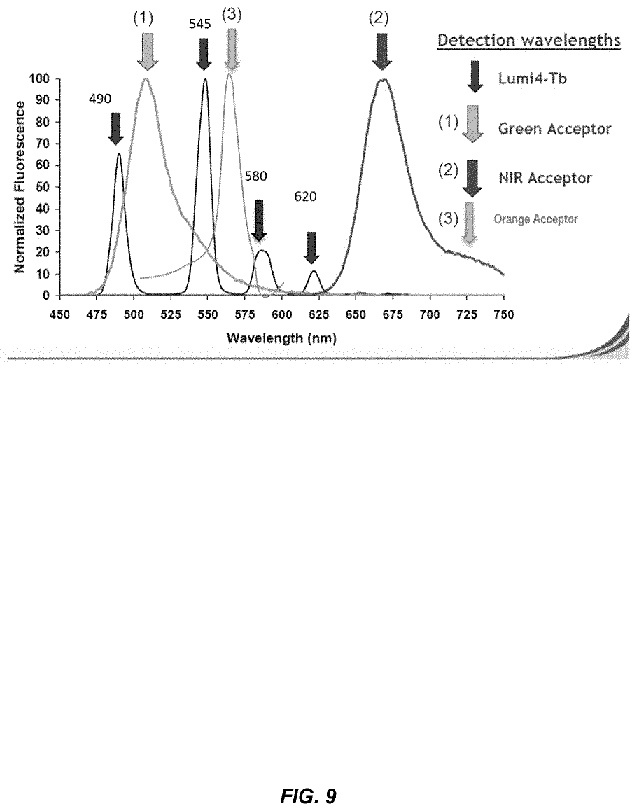

The acceptor molecules that can be used include, but are not limited to, fluorescein-like (green zone) acceptor, Cy5, DY-647, Alexa Fluor 488, Alexa Fluor 546, Allophycocyanin (APC), Phycoeruythrin (PE) and Alexa Fluor 647 (FIG. 9). Donor and acceptor fluorophores having reactive moieties such as an NHS ester can be conjugated using a primary amine on an protein or antibody.

Other acceptors include, but are not limited to, cyanin derivatives, D2, CY5, fluorescein, coumarin, rhodamine, carbopyronine, oxazine and its analogs, Alexa Fluor fluorophores, Crystal violet, perylene bisimide fluorophores, squaraine fluorophores, boron dipyrromethene derivatives, NBD (nitrobenzoxadiazole) and its derivatives, DABCYL (4-((4-(dimethylamino)phenyl)azo)benzoic acid).

In one aspect, fluorescence can be characterized by wavelength, intensity, lifetime, polarization or a combination thereof.

3. Antibodies

In certain aspects, an activated ester (an NHS ester) of the donor or acceptor can react with a primary amine on an antibody to make a stable amide bond. For example, a maleimide on the cryptate or the acceptor (e.g., Alexa 647) and a thiol on the antibody can react together and make a thioether. Alkyl halides react with amines and thiols to make alkylamines and thioethers, respectively. Any derivative providing a reactive moiety that can be conjugated to an antibody can be utilized herein to make the first antibody labeled with a donor fluorophore specific for the analyte, as well as, the second antibody labeled with an acceptor fluorophore specific for analyte.

The methods herein can use a variety of samples, which include a tissue sample, blood, biopsy, serum, plasma, saliva, urine, or stool sample.

4. Production of Antibodies

The generation and selection of antibodies not already commercially available can be accomplished several ways. For example, one way is to express and/or purify a polypeptide of interest (i.e., antigen) using protein expression and purification methods known in the art, while another way is to synthesize the polypeptide of interest using solid phase peptide synthesis methods known in the art. See, e.g., Guide to Protein Purification, Murray P. Deutcher, ed., Meth. Enzymol., Vol. 182 (1990); Solid Phase Peptide Synthesis, Greg B. Fields, ed., Meth. Enzymol., Vol. 289 (1997); Kiso et al., Chem. Pharm. Bull., 38:1192-99 (1990); Mostafavi et al., Biomed. Pept. Proteins Nucleic Acids, 1:255-60, (1995); and Fujiwara et al., Chem. Pharm. Bull., 44:1326-31 (1996). The purified or synthesized polypeptide can then be injected, for example, into mice or rabbits, to generate polyclonal or monoclonal antibodies. One skilled in the art will recognize that many procedures are available for the production of antibodies, for example, as described in Antibodies, A Laboratory Manual, Harlow and Lane, Eds., Cold Spring Harbor Laboratory, Cold Spring Harbor, N.Y. (1988). One skilled in the art will also appreciate that binding fragments or Fab fragments which mimic antibodies can also be prepared from genetic information by various procedures (see, e.g., Antibody Engineering: A Practical Approach, Borrebaeck, Ed., Oxford University Press, Oxford (1995); and Huse et al., J. Immunol., 149:3914-3920 (1992)).

In addition, numerous publications have reported the use of phage display technology to produce and screen libraries of polypeptides for binding to a selected target antigen (see, e.g, Cwirla et al., Proc. Natl. Acad. Sci. USA, 87:6378-6382 (1990); Devlin et al., Science, 249:404-406 (1990); Scott et al., Science, 249:386-388 (1990); and Ladner et al., U.S. Pat. No. 5,571,698). A basic concept of phage display methods is the establishment of a physical association between a polypeptide encoded by the phage DNA and a target antigen. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target antigen bind to the target antigen and these phage are enriched by affinity screening to the target antigen. The identity of polypeptides displayed from these phage can be determined from their respective genomes. Using these methods, a polypeptide identified as having a binding affinity for a desired target antigen can then be synthesized in bulk by conventional means (see, e.g., U.S. Pat. No. 6,057,098).

The antibodies that are generated by these methods can then be selected by first screening for affinity and specificity with the purified polypeptide antigen of interest and, if required, comparing the results to the affinity and specificity of the antibodies with other polypeptide antigens that are desired to be excluded from binding. The screening procedure can involve immobilization of the purified polypeptide antigens in separate wells of microtiter plates. The solution containing a potential antibody or group of antibodies is then placed into the respective microtiter wells and incubated for about 30 minutes to 2 hours. The microtiter wells are then washed and a labeled secondary antibody (e.g., an anti-mouse antibody conjugated to alkaline phosphatase if the raised antibodies are mouse antibodies) is added to the wells and incubated for about 30 minutes and then washed. Substrate is added to the wells and a color reaction will appear where antibody to the immobilized polypeptide antigen is present.

The antibodies so identified can then be further analyzed for affinity and specificity. In the development of immunoassays for a target protein (e.g, S-protein or N-protein), the purified target protein acts as a standard with which to judge the sensitivity and specificity of the immunoassay using the antibodies that have been selected. Because the binding affinity of various antibodies may differ, e.g., certain antibody combinations may interfere with one another sterically, assay performance of an antibody may be a more important measure than absolute affinity and specificity of that antibody.

Those skilled in the art will recognize that many approaches can be taken in producing antibodies or binding fragments and screening and selecting for affinity and specificity for the various polypeptides of interest, but these approaches do not change the scope of the present invention.

A. Polyclonal Antibodies

Polyclonal antibodies are preferably raised in animals by multiple subcutaneous (sc) or intraperitoneal (ip) injections of a polypeptide of interest and an adjuvant. It may be useful to conjugate the polypeptide of interest to a protein carrier that is immunogenic in the species to be immunized, such as, e.g., keyhole limpet hemocyanin, serum albumin, bovine thyroglobulin, or soybean trypsin inhibitor using a bifunctional or derivatizing agent. Non-limiting examples of bifunctional or derivatizing agents include maleimidobenzoyl sulfosuccinimide ester (conjugation through cysteine residues), N-hydroxysuccinimide (conjugation through lysine residues), glutaraldehyde, succinic anhydride, SOCl₂, and R₁N═C═NR, wherein R and R₁ are different alkyl groups.

Animals are immunized against the polypeptide of interest or an immunogenic conjugate or derivative thereof by combining, e.g., 100 μg (for rabbits) or 5 μg (for mice) of the antigen or conjugate with 3 volumes of Freund's complete adjuvant and injecting the solution intradermally at multiple sites. One month later, the animals are boosted with about ⅕ to 1/10 the original amount of polypeptide or conjugate in Freund's incomplete adjuvant by subcutaneous injection at multiple sites. Seven to fourteen days later, the animals are bled and the serum is assayed for antibody titer. Animals are typically boosted until the titer plateaus. Preferably, the animal is boosted with the conjugate of the same polypeptide, but conjugation to a different immunogenic protein and/or through a different cross-linking reagent may be used. Conjugates can also be made in recombinant cell culture as fusion proteins. In certain instances, aggregating agents such as alum can be used to enhance the immune response.

B. Monoclonal Antibodies

Monoclonal antibodies are generally obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies. For example, monoclonal antibodies can be made using the hybridoma method described by Kohler et al., Nature, 256:495 (1975) or by any recombinant DNA method known in the art (see, e.g., U.S. Pat. No. 4,816,567).

In the hybridoma method, a mouse or other appropriate host animal (e.g., hamster) is immunized as described above to elicit lymphocytes that produce or are capable of producing antibodies which specifically bind to the polypeptide of interest used for immunization. Alternatively, lymphocytes are immunized in vitro. The immunized lymphocytes are then fused with myeloma cells using a suitable fusing agent, such as polyethylene glycol, to form hybridoma cells (see, e.g., Goding, Monoclonal Antibodies: Principles and Practice, Academic Press, pp. 59-103 (1986)). The hybridoma cells thus prepared are seeded and grown in a suitable culture medium that preferably contains one or more substances which inhibit the growth or survival of the unfused, parental myeloma cells. For example, if the parental myeloma cells lack the enzyme hypoxanthine guanine phosphoribosyl transferase (HGPRT), the culture medium for the hybridoma cells will typically include hypoxanthine, aminopterin, and thymidine (HAT medium), which prevent the growth of HGPRT-deficient cells.

Preferred myeloma cells are those that fuse efficiently, support stable high-level production of antibody by the selected antibody-producing cells, and/or are sensitive to a medium such as HAT medium. Examples of such preferred myeloma cell lines for the production of human monoclonal antibodies include, but are not limited to, murine myeloma lines such as those derived from MOPC-21 and MPC-11 mouse tumors (available from the Salk Institute Cell Distribution Center; San Diego, Calif.), SP-2 or X63-Ag8-653 cells (available from the American Type Culture Collection; Rockville, Md.), and human myeloma or mouse-human heteromyeloma cell lines (see, e.g., Kozbor, J. Immunol., 133:3001 (1984); and Brodeur et al., Monoclonal Antibody Production Techniques and Applications, Marcel Dekker, Inc., New York, pp. 51-63 (1987)).

The culture medium in which hybridoma cells are growing can be assayed for the production of monoclonal antibodies directed against the polypeptide of interest. Preferably, the binding specificity of monoclonal antibodies produced by hybridoma cells is determined by immunoprecipitation or by an in vitro binding assay, such as a radioimmunoassay (RIA) or an enzyme-linked immunoabsorbent assay (ELISA). The binding affinity of monoclonal antibodies can be determined using, e.g., the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (see, e.g., Goding, Monoclonal Antibodies: Principles and Practice, Academic Press, pp. 59-103 (1986)). Suitable culture media for this purpose include, for example, D-MEM or RPMI-1640 medium. In addition, the hybridoma cells may be grown in vivo as ascites tumors in an animal. The monoclonal antibodies secreted by the subclones can be separated from the culture medium, ascites fluid, or serum by conventional antibody purification procedures such as, for example, protein A-Sepharose, hydroxylapatite chromatography, gel electrophoresis, dialysis, or affinity chromatography.

DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody, to induce the synthesis of monoclonal antibodies in the recombinant host cells. See, e.g., Skerra et al., Curr. Opin. Immunol., 5:256-262 (1993); and Pluckthun, Immunol Rev., 130:151-188 (1992). The DNA can also be modified, for example, by substituting the coding sequence for human heavy chain and light chain constant domains in place of the homologous murine sequences (see, e.g., U.S. Pat. No. 4,816,567; and Morrison et al., Proc. Natl. Acad. Sci. USA, 81:6851 (1984)), or by covalently joining to the immunoglobulin coding sequence all or part of the coding sequence for a non-immunoglobulin polypeptide.

In a further embodiment, monoclonal antibodies or antibody fragments can be isolated from antibody phage libraries generated using the techniques described in, for example, McCafferty et al., Nature, 348:552-554 (1990); Clackson et al., Nature, 352:624-628 (1991); and Marks et al., J. Mol. Biol., 222:581-597 (1991). The production of high affinity (nM range) human monoclonal antibodies by chain shuffling is described in Marks et al., BioTechnology, 10:779-783 (1992). The use of combinatorial infection and in vivo recombination as a strategy for constructing very large phage libraries is described in Waterhouse et al., Nuc. Acids Res., 21:2265-2266 (1993). Thus, these techniques are viable alternatives to traditional monoclonal antibody hybridoma methods for the generation of monoclonal antibodies. Human Antibodies

As an alternative to humanization, human antibodies can be generated. In some embodiments, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immun., 7:33 (1993); and U.S. Pat. Nos. 5,591,669, 5,589,369, and 5,545,807.

Alternatively, phage display technology (see, e.g., McCafferty et al., Nature, 348:552-553 (1990)) can be used to produce human antibodies and antibody fragments in vitro, using immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats as described in, e.g., Johnson et al., Curr. Opin. Struct. Biol., 3:564-571 (1993). Several sources of V-gene segments can be used for phage display. See, e.g., Clackson et al., Nature, 352:624-628 (1991). A repertoire of V genes from unimmunized human donors can be constructed and antibodies to a diverse array of antigens (including self-antigens) can be isolated essentially following the techniques described in Marks et al., J. Mol. Biol., 222:581-597 (1991); Griffith et al., EMBO J., 12:725-734 (1993); and U.S. Pat. Nos. 5,565,332 and 5,573,905.

In certain instances, human antibodies can be generated by in vitro activated B cells as described in, e.g., U.S. Pat. Nos. 5,567,610 and 5,229,275.

C. Antibody Fragments

Various techniques have been developed for the production of antibody fragments. Traditionally, these fragments were derived via proteolytic digestion of intact antibodies (see, e.g., Morimoto et al., J. Biochem. Biophys. Meth., 24:107-117 (1992); and Brennan et al., Science, 229:81 (1985)). However, these fragments can now be produced directly using recombinant host cells. For example, the antibody fragments can be isolated from the antibody phage libraries discussed above. Alternatively, Fab′-SH fragments can be directly recovered from E. coli cells and chemically coupled to form F(ab′)2 fragments (see, e.g., Carter et al., BioTechnology, 10:163-167 (1992)). According to another approach, F(ab′)2 fragments can be isolated directly from recombinant host cell culture. Other techniques for the production of antibody fragments will be apparent to those skilled in the art. In other embodiments, the antibody of choice is a single chain Fv fragment (scFv). See, e.g., PCT Publication No. WO 93/16185; and U.S. Pat. Nos. 5,571,894 and 5,587,458. The antibody fragment may also be a linear antibody as described, e.g., in U.S. Pat. No. 5,641,870. Such linear antibody fragments may be monospecific or bispecific.

D. Bispecific Antibodies

Bispecific antibodies are antibodies that have binding specificities for at least two different epitopes. Exemplary bispecific antibodies may bind to two different epitopes of the same polypeptide of interest. Other bispecific antibodies may combine a binding site for the polypeptide of interest with binding site(s) for one or more additional antigens. Bispecific antibodies can be prepared as full-length antibodies or antibody fragments (e.g., F(ab′)2 bispecific antibodies).

Methods for making bispecific antibodies are known in the art. Traditional production of full-length bispecific antibodies is based on the co-expression of two immunoglobulin heavy chain-light chain pairs, where the two chains have different specificities (see, e.g., Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule is usually performed by affinity chromatography. Similar procedures are disclosed in PCT Publication No. WO 93/08829 and Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy chain constant region (CH1) containing the site necessary for light chain binding present in at least one of the fusions. DNA encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains into one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. See, e.g., PCT Publication No. WO 94/04690 and Suresh et al., Meth. Enzymol., 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the CH3 domain of an antibody constant domain. In this method, one or more small amino acid side-chains from the interface of the first antibody molecule are replaced with larger side chains (e.g., tyrosine or tryptophan). Compensatory “cavities” of identical or similar size to the large side-chain(s) are created on the interface of the second antibody molecule by replacing large amino acid side-chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies can be made using any convenient cross-linking method. Suitable cross-linking agents and techniques are well-known in the art, and are disclosed in, e.g., U.S. Pat. No. 4,676,980.

Suitable techniques for generating bispecific antibodies from antibody fragments are also known in the art. For example, bispecific antibodies can be prepared using chemical linkage. In certain instances, bispecific antibodies can be generated by a procedure in which intact antibodies are proteolytically cleaved to generate F(ab′)2 fragments (see, e.g., Brennan et al., Science, 229:81 (1985)). These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody.

In some embodiments, Fab′-SH fragments can be directly recovered from E. coli and chemically coupled to form bispecific antibodies. For example, a fully humanized bispecific antibody F(ab′)2 molecule can be produced by the methods described in Shalaby et al., J. Exp. Med., 175: 217-225 (1992). Each Fab′ fragment was separately secreted from E. coli and subjected to directed chemical coupling in vitro to form the bispecific antibody.

Various techniques for making and isolating bispecific antibody fragments directly from recombinant cell culture have also been described. For example, bispecific antibodies have been produced using leucine zippers. See, e.g., Kostelny et al., J. Immunol., 148:1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy chain variable domain (VH) connected to a light chain variable domain (VL) by a linker which is too short to allow pairing between the two domains on the same chain. Accordingly, the VH and VL domains of one fragment are forced to pair with the complementary VL and VH domains of another fragment, thereby forming two antigen binding sites. Another strategy for making bispecific antibody fragments by the use of single-chain Fv (sFv) dimers is described in Gruber et al., J. Immunol., 152:5368 (1994).

Antibodies with more than two valencies are also contemplated. For example, trispecific antibodies can be prepared. See, e.g., Tutt et al., J. Immunol., 147:60 (1991).

E. Antibody Purification

When using recombinant techniques, antibodies can be produced inside an isolated host cell, in the periplasmic space of a host cell, or directly secreted from a host cell into the medium. If the antibody is produced intracellularly, the particulate debris is first removed, for example, by centrifugation or ultrafiltration. Carter et al., BioTech., 10:163-167 (1992) describes a procedure for isolating antibodies which are secreted into the periplasmic space of E. coli. Briefly, cell paste is thawed in the presence of sodium acetate (pH 3.5), EDTA, and phenylmethylsulfonylfluoride (PMSF) for about 30 min. Cell debris can be removed by centrifugation. Where the antibody is secreted into the medium, supernatants from such expression systems are generally concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from cells can be purified using, for example, hydroxylapatite chromatography, gel electrophoresis, dialysis, and affinity chromatography. The suitability of protein A as an affinity ligand depends on the species and isotype of any immunoglobulin Fc domain that is present in the antibody. Protein A can be used to purify antibodies that are based on human γ1, γ2, or γ4 heavy chains (see, e.g., Lindmark et al., J. Immunol. Meth., 62:1-13 (1983)). Protein G is recommended for all mouse isotypes and for human γ3 (see, e.g., Guss et al., EMBO J., 5:1567-1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker; Phillipsburg, N.J.) is useful for purification. Other techniques for protein purification such as fractionation on an ion-exchange column, ethanol precipitation, reverse phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25 M salt).

### III. Specific Antibodies

In one aspect, an Anti-2019-nCov Spike Protein S2 Domain mAb (COVID 19 S2 Protein Coronavirus (COVID-19), Monoclonal Antibody), which has an IgG isotype, cat no. MBS8574747, which is available from Mybiosouce.com, can be used in the methods of the present invention. Alternatively, the COVID 19 S1 Protein Humanized Coronavirus, Monoclonal Antibody (#MBS355889); or the COVID 19 S2 Protein (S2a) Coronavirus, Antibody (#MBS8574748), or COVID 19 Spike glycoprotein (S) Coronavirus, Monoclonal Recombinant Antibody (#MBS7135928), all available from Mybiosouce.com, can be used.

In another aspect, COVID 19 Coronavirus, Monoclonal Antibody (#MBS569937), Coronavirus (COVID-19 & SARS-CoV NP) Antibody, which has COVID-19 & SARS Coronavirus Nucleoprotein (NP) specificity can be used.

In still another aspect, COVID 19 N (BN18) Coronavirus (COVID-19), Monoclonal Antibody, Human anti-2019-nCoV N mAb (BN18), which binds to N-terminus of 2019-nCoV NP, cat. No. MBS8574744, available from Mybiosouce.com, can be used in the methods of the present invention.

In addition, antibodies to SARS-CoV-2 are available from ProSci, 12170 Flint Place Poway, Calif. 92064, USA. The SARS-CoV-2/SARS-CoV Spike antibody (Cat. No. 3221) can be used for the detection of SARS-CoV-2/SARS-CoV Spike protein. In another aspect, the Anti-SARS-CoV Spike antibody (Cat. No. 3225), which was raised against a peptide corresponding to 15 amino acids near the center of SARS-CoV Spike glycoprotein, can also be used. The immunogen is located within amino acids 650-700 of SARS-CoV Spike.

In addition, in certain instances, goat anti-Human IgM, Polyclonal Antibody is available from mybiosource.com and has catalogue number MBS560396.

### IV. Kits

In one embodiment, the present disclosure provides a kit for the detection of antibodies induced by SARS-CoV-2 (anti-SARS-CoV-2) in a biological sample from a subject, comprising:
a lyophilized first labeled protein with a donor fluorophore; and
a lyophilized second labeled protein with an acceptor fluorophore, wherein the first and second proteins are both spike proteins (S-protein) or wherein the first and second proteins are both nucleocapsid proteins (N-proteins).

In certain aspects, the S-protein is a mammalian cell expressed recombinant spike protein of SARS-CoV-2 or fragment thereof.

In certain aspects, the N-protein is a mammalian cell expressed recombinant nucleocapsid protein of SARS-CoV-2 or fragment thereof.

In certain aspects, the cryptate is a terbium cryptate.

In certain aspects, the acceptor fluorophore is selected from the group consisting of fluorescein-like (green zone) dyes, Cy5, DY-647, phycoerythrin, allophycocyanin (APC), Alexa Fluor 488, Alexa Fluor 546, and Alexa Fluor 647.

In certain aspects, the kit further comprises instructions for use.

In certain aspects, the kit further comprises a cuvette having lyophilized reagents.

### V. Devices

Various instruments and devices are suitable for use in the present disclosure. Many spectrophotometers have the capability to measure fluorescence. Fluorescence is the molecular absorption of light energy at one wavelength and its nearly instantaneous re-emission at another, longer wavelength. Some molecules fluoresce naturally, and others must be modified to fluoresce.

A fluorescence spectrophotometer or fluorometer, fluorospectrometer, or fluorescence spectrometer measures the fluorescent light emitted from a sample at different wavelengths, after illumination with light source such as a xenon flash lamp. Fluorometers can have different channels for measuring differently-colored fluorescent signals (that differ in their wavelengths), such as green and blue, or ultraviolet and blue, channels. In one aspect, a suitable device includes an ability to perform a time-resolved fluorescence resonance energy transfer (FRET) experiment.

Suitable fluorometers can hold samples in different ways, including cuvettes, capillaries, Petri dishes, and microplates. The assays described herein can be performed on any of these types of instruments. In certain aspects, the device has an optional microplate reader, allowing emission scans in up to 384-well plates. Others models suitable for use hold the sample in place using surface tension.

Suitable plate readers include a ClarioSTAR plate reader (BMG Labtech, Cary, N.C.) or an Infinite 200 PRO (Tecan Group Ltd, Mannedorf, Switzerland). These devices can detect one or more of parameters including absorbance, luminescence, fluorescence, and the like. In certain aspects, the photometric, spectrophotometric, or fluorescent measurement is performed by a plate reader (also known as a microplate reader). Plate readers permit high throughput measurement on one or a plurality of samples (e.g., at least 1, at least 2, at least 5, at least 10, at least 15, at least 20, at least 25, at least 30, at least 50, at least 100, or at least 200). Plate readers are commercially available through companies such as Tecan, Molecular Devices, Thermo Scientific, BioTek, and Bio-Rad.

Time-resolved fluorescence (TRF) measurement is similar to fluorescence intensity measurement. One difference, however, is the timing of the excitation/measurement process. When measuring fluorescence intensity, the excitation and emission processes are simultaneous: the light emitted by the sample is measured while excitation is taking place. Even though emission systems are very efficient at removing excitation light before it reaches the detector, the amount of excitation light compared to emission light is such that fluorescent intensity measurements exhibit elevated background signals. The present disclosure offers a solution to this issue. Time resolve FRET relies on the use of specific fluorescent molecules that have the property of emitting over long periods of time (measured in milliseconds) after excitation, when most standard fluorescent dyes (e.g. fluorescein) emit within a few nanoseconds of being excited. As a result, it is possible to excite cryptate lanthanides using a pulsed light source (e.g., Xenon flash lamp or pulsed laser), and measure after the excitation pulse.

As the donor and acceptor fluorescent compounds attached to antibody 1 and 2 move closer together, an energy transfer is caused from the donor compound to the acceptor compound, resulting in a decrease in the fluorescence signal emitted by the donor compound and an increase in the signal emitted by the acceptor compound, and vice-versa. The majority of biological phenomena involving interactions between different partners will therefore be able to be studied by measuring the change in FRET between 2 fluorescent compounds coupled with compounds which will be at a greater or lesser distance, depending on the biological phenomenon in question.

The FRET signal can be measured in different ways: measurement of the fluorescence emitted by the donor alone, by the acceptor alone or by the donor and the acceptor, or measurement of the variation in the polarization of the light emitted in the medium by the acceptor as a result of FRET. One can also include measurement of FRET by observing the variation in the lifetime of the donor, which is facilitated by using a donor with a long fluorescence lifetime, such as rare earth complexes (especially on simple equipment like plate readers). Furthermore, the FRET signal can be measured at a precise instant or at regular intervals, making it possible to study its change over time and thereby to investigate the kinetics of the biological process studied.

In certain aspects, the device disclosed in PCT/IB2019/051213, filed Feb. 14, 2019 is used, which is hereby incorporated by reference. That disclosure in that application generally relates to analyzers that can be used in point-of-care (POC) settings to measure the absorbance and fluorescence of a sample with minimal or no user handling or interaction. The disclosed analyzers provide advantageous features of more rapid and reliable analyses of samples having properties that can be detected with each of these two approaches. For example, it can be beneficial to quantify both the fluorescence and absorbance of a blood sample being subjected to a diagnostic assay. In some analytical workflows, the hematocrit of a blood sample can be quantified with an absorbance assay, while the signal intensities measured in a FRET assay can provide information regarding other components of the blood sample.

One apparatus disclosed in PCT/IB2019/051213 is useful for detecting an emission light from a sample, and absorbance of a transillumination light by the sample, which comprises a first light source configured to emit an excitation light having an excitation wavelength. The apparatus further comprises a second light source configured to transilluminate the sample with the transillumination light. The apparatus further comprises a first light detector configured to detect the excitation light, and a second light detector configured to detect the emission light and the transillumination light. The apparatus further comprises a dichroic mirror configured to (1) epi-illuminate the sample by reflecting at least a portion of the excitation light, (2) transmit at least a portion of the excitation light to the first light detector, (3) transmit at least a portion of the emission light to the second light detector, and (4) transmit at least a portion of the transillumination light to the second light detector.

One suitable cuvette for use in the present disclosure is disclosed in PCT/IB2019/051215, filed Feb. 14, 2019. One of the provided cuvettes comprises a hollow body enclosing an inner chamber having an open chamber top. The cuvette further comprises a lower lid having an inner wall, an outer wall, an open lid top, and an open lid bottom. At least a portion of the lower lid is configured to fit inside the inner chamber proximate to the open chamber top. The lower lid comprises one or more (e.g., two or more) containers connected to the inner wall, wherein each of the containers has an open container top. In certain aspects, the lower lid comprises two or more such containers. The lower lid further comprises a securing means connected to the hollow body. The cuvette further comprises an upper lid wherein at least a portion of the upper lid is configured to fit inside the lower lid proximate to the open lid top.

### VI. Examples

### Example 1

Detection of SARS COV-2 IgG by a Commercial ELISA Kit.

This example uses a commercially available ELISA kit (Coronavirus COVID-19 IgG ELISA Assay; SKU KT-1032, Eagle Biosciences, Amherst N.H.) for the detection of IgG antibodies. The results are compared to assays of the current disclosure.

Assay controls and test samples are added to microtiter wells of a microplate that are coated with the COVID-19 peptide antigen nucleocapsid protein. After the first incubation period, the unbound protein matrix is removed with a subsequent washing step. A horseradish peroxidase labeled COVID-19 IgG tracer antibody is added to each well. After an incubation period, an immunocomplex of “COVID-19 polypeptide antigen-new coronavirus IgG antibody HRP labeled COVID-19 IgG tracer antibody” is formed if there is coronavirus IgG antibody present in the tested materials. The unbound tracer antibody is removed by the subsequent washing step. HRP labeled tracer antibody bound to the well is then incubated with a substrate solution in a timed reaction and then measured in a spectrophotometric microplate reader. The enzymatic activity of the tracer antibody bound to the coronavirus IgG on the wall of the microtiter well is proportional to the amount of the coronavirus IgG antibody level in the test sample.

### Example 2

Detection of SARS COV-2 IgM by a Commercial ELISA Kit

This example uses a commercially available ELISA kit (Coronavirus COVID-19 IgM ELISA Assay; SKU KT-1033, Eagle Biosciences, Amherst N.H.) for the detection of IgM antibodies. The results are compared to assays of the current disclosures.

Assay controls, test samples and biotinylated COVID-19 specific peptide antigens are added to the microtiter wells of a microplate that is coated with an anti-human IgM specific antibody. This assay utilizes the “IgM capture” method on microplate based enzyme immunoassay technique After the first incubation period, the unbound protein matrix is removed with a subsequent washing step. A horseradish peroxidase (HRP) labeled streptavidin is added to each well. After an incubation period, an immunocomplex of “Anti-hIgM antibody-human nCoV IgM antibody-HRP labeled COVID-19 antigen” is formed if there is novel coronavirus IgM antibody present in the tested materials. The unbound tracer antibody is removed by the subsequent washing step. HRP-labeled COVID-19 antigen tracer bound to the well is then incubated with a substrate solution in a timed reaction and is then measured in a spectrophotometric microplate reader. The enzymatic activity of the tracer antibody bound to the coronoavirus IgM on the wall of the microtiter well is proportional to the amount of the coronavirus IgM antibody level in the test sample.

### Example 3

This example uses a solution phase bridging assay for detecting antibodies induced by SARS-CoV-2 (anti-SARS-CoV-2) in a serum sample.

In this assay, the assay includes contacting the sample with a first labeled protein with a donor fluorophore. The first protein is a COVID 19 Spike Protein RBD Coronavirus, Recombinant Protein (#MBS8574751) from Mybiosource.com. The first protein is labeled with a terbium cryptate donor. The sample is then contacted with a second labeled protein with an acceptor fluorophore, the second protein is a COVID 19 Spike Protein RBD Coronavirus, Recombinant Protein (#MBS8574751) from Mybiosource.com. The second protein is labeled with Alexa Fluor 647.

The sample is allowed to incubate for a time sufficient to generate a ternary complex of the first labeled protein with a donor fluorophore, the second labeled protein labeled with an acceptor fluorophore and the anti-SARS-CoV-2. The sample is excited using a light source to detect a fluorescence emission signal associated with fluorescence resonance energy transfer (FRET) when the donor fluorophore is excited. The presence of antibodies in the sample bind both proteins in a bridging assay leading to a concentration-dependent increase in fluorescence emission. The level of light emitted from the acceptor fluorophore is proportional to the degree of donor/acceptor complex formation (the ternary complex).

### Example 4

This example illustrates a competitive assay method for detecting antibodies induced by SARS-CoV-2 (anti-SARS-CoV-2) in a biological sample from a subject.

In this assay, a sample is contacted with a complex comprising an anti-SARS-CoV-2 antibody (COVID-19 Spike glycoprotein (S) Coronavirus, Monoclonal Recombinant Antibody (#MBS7135928)) labeled with a terbium cryptate donor.

In addition, an isolated labeled protein(s) with a second fluorophore (a COVID 19 Spike Protein RBD Coronavirus, Recombinant Protein (#MBS8574751) from Mybiosource.com, labeled with Alexa Fluor 647).

The complex emits a fluorescence emission signal associated with fluorescence resonance energy transfer (FRET) when the first fluorophore is excited using a light source. The complex is incubated for a time sufficient for the anti-SARS-CoV-2 in the sample to compete for binding with the anti-SARS-CoV-2 antibody labeled with the first fluorophore

The sample is excited using a light source to detect the fluorescence emission signal associated with FRET, wherein a decrease in the fluorescence emission signal relative to the fluorescence emission signal initially emitted by the complex indicates the presence or amount of antibodies induced by SARS-CoV-2 (anti-SARS-CoV-2) in the sample.

### Example 5

This example illustrates a SARS COV-2 Ab a qualitative time resolved fluorescence immunoassay kit for the detection of antibodies to SARS COV-2 in human serum or plasma.

The SARS COV-2 Ab is an time resolved fluorescence resonance energy (trFRET) immunoassay for the detection of IgG and IgM antibodies against the SARS COV-2 S protein using on a high throughput plate reader (e.g. Tecan Spark) which can detect in human serum or plasma within 10 minutes.

Fluorescence resonance energy transfer (FRET) is a process in which a donor fluorescent molecule, in an excited state, transfers excitation energy to an acceptor fluorophore when the two are brought into close proximity (˜100 Å). Upon excitation at a characteristic wavelength the energy absorbed by the donor is transferred to the acceptor, which in turn emits light energy. The level of light emitted from the acceptor fluorophore is proportional to the degree of donor/acceptor complex formation. When the specific binding event does not occur, no additional FRET signal is present.

The tr fluorescent plate reader detects time-resolved (tr) fluorescent signal from both the donor and FRET acceptor emission. Time-resolved (tr) is a technique to improve signal to noise by removing short-lived fluorescent signals originating from the sample. The donor fluorophore is excited using a pulse of light. The emission from both the donor and acceptor signals are read after a time delay from the end of the excitation pulse. Noise is reduced as background fluorescence from nonspecific sources decay more rapidly than the emitted light from the donor allowing the acceptor signal to be read long after the nonspecific fluorescence has passed.

The SARS COV-2 Ab assay uses a donor fluorophore consisting of Terbium bound within a cryptate. The fluorescent reader excites the Terbium cryptate with a 365 nm UV LED. The Terbium cryptate emits at four (4) wavelengths within the visible region. The assay uses the lowest donor emission energy peak of 620 nm as the donor signal within the assay. The acceptor fluorophore, when in very close proximity, is excited by the highest energy Terbium cryptate emission peak of 490 nm causing light emission at 520 nm. Both the 620 nm and 520 nm emission wavelengths are measured independently in the instrument and results are reported as RFU ratio 620/520.

Recombinant SARS COV-2 S protein is labeled with Tb cryptate and in a separate reaction labeled with fluorescent acceptor. The lyophilized fluorescent conjugates contain S molecules labelled only with Tb cryptate (donor) and S molecules labelled only with fluorescent donor. When one arm of the IgG or IgM molecule in patient specimen binds to S protein acceptor and the other arm of the same antibody binds to the S protein donor then a tr FRET signal is generated. This is often referred to a solution phase bridging assay. The trFRET signal is directly proportional to the concentration of anti-SARS S antibody in the serum or plasma.

### Contents of the Kit

R1 Microplate 1 plate; 12 strips of 8 wells containing lyophilized fluorescently labelled purified SARS COV-2 antigens.

R2 Negative Control 1 vial; Heat inactivated human plasma negative for SARS Ab, (2.5 ml). Preservative: Sodium azide<0.1%.

R3 Ab Positive control 1 vial; Heat inactivated human plasma positive for anti-SARS (1 ml) antibodies, negative for HIV and HBs antigens, in synthetic diluent; Preservative: ProClin™ 300<0.1%.

Reagent 1 (R1): Microplate. Each frame support containing 12 strips is wrapped in a sealed foil bag. Cut the bag using scissors 0.5 to 1 cm above the sealing. Open the bag and take out the frame. Put the unused strips back into the bag. Close the bag carefully and put it back into storage at +2-8° C.

Reagent 2 (R2): Negative Control

Reagent 3 (R3): SARS Ab Positive Control

The kit should be stored at +2-8° C. When stored at this temperature, each reagent contained in the Procise™ SARS COV-2 Ab can be used until the expiry date on the kit.

R1: After the vacuum-sealed bag has been opened, the microwell strips stored at +2-8° C. in the carefully resealed bag can be used for 1 month.

R2 and R3: The reagents stored at +2-8° C. can be used for 4 weeks after the vials have been reconstituted.

Collect a blood sample according to the current practices. The test should be performed on undiluted serum or plasma (collected with EDTA, heparin, citrate, ACD-based anticoagulants). Separate the serum or plasma from the clot or red cells as soon as possible to avoid any hemolysis. Extensive hemolysis may affect test performance. Specimens with observable particulate matter should be clarified by centrifugation prior testing. Suspended fibrin particles or aggregates may yield falsely positive results.

Do not heat the samples.

The specimens can be stored at +2-8° C. if screening is performed within 7 days or they may be frozen at −20° C. for several months. The plasma must be quickly thawed by warming for a few minutes in a water bath at 40° C. (To avoid fibrin precipitation). Do not repeat more than 3 freeze/thaw cycles.

If the specimens are to be shipped, they must be packaged in accordance with the regulations in force regarding the transport of etiological agents.

Use the negative (R2), SARS Ab positive (R3) controls for each assay run as these are used to calculate the assay cutoff
1. Take the carrier tray and the strips (R1) out of the protective pouch,
2. Add 100 μl of SARS Ab positive control (R2) in well A1-A2
3. Add 100 μl of Negative control (R3) in well C1, D1 and E1
4. Add 100 μl of specimen 1 in well F1
5. Add 100 μl specimen 2 in well GI, and so on
6. Tap to mix or mix on a plate shaker
7. Cover the microplate with adhesive film. Press firmly all over the plate to ensure a tight seal.
8. Incubate the microplate RT for 5 min—agitation or heat at 37
9. After 5 min RT incubation place the microtiter plate in the Tecan SPARK Instrument.
10. After reading the plate dispose of the plate in biohazard waste. Do not remove the adhesive film.

The presence or absence of detectable SARS COV-2 Antigen or antibodies to SARS COV-2 is determined by comparing the tr fluorescence measured for each sample to the calculated cut-off value.

Samples with RFU values less than the cut-off value are considered to be negative by the SARS COV-2 Ab test.

### Example 6

An immunoassay (ELISA) was carried out to investigate the binding of a sample containing antibodies to an N peptide by using a 4-P6 peptide (SEQ ID NO:34) coated plate. The peptide has the following sequence: NH₂-TQALPQRQKKQQTVTLLPAADLDDFSK-OH. The plate was coated with diluted peptide and blocking buffer was added. After shaking, the plates were washed 3-times in PBS-Tween (0.05%). The sample was added and incubated for 1 hour with shaking. Thereafter the sample was washed and an anti-human IgG-HRP diluted 1:5000 in PBS 1% BSA was added and incubated for 1 hour. Excess sample was washed and one-step substrate was added and develop until a blue color is detectable but not saturated (5-15 min). A stop solution was added and absorbance read at 450 nm.

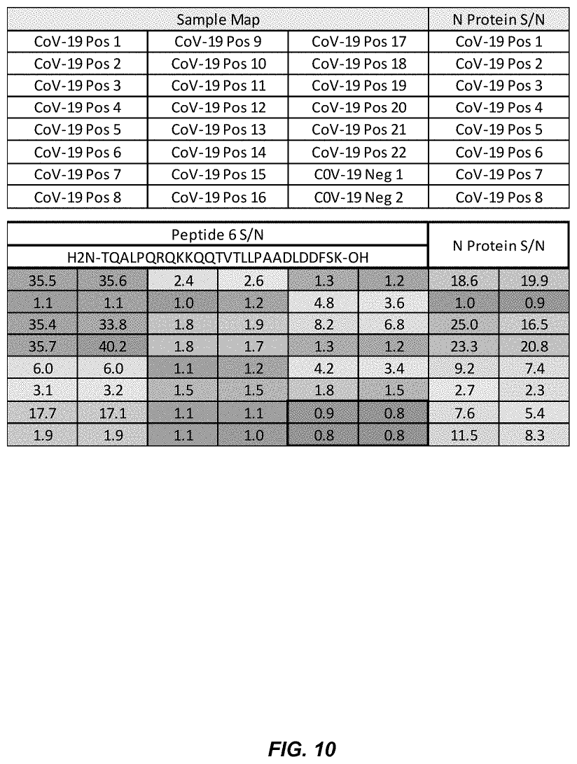

FIG. 10 shows the ELISA site map and the signal over noise of the positive samples. Positions 23 and 24 were negative controls.

This assay can be performed in a competitive assay format for detecting antibodies induced by SARS-CoV-2 (anti-SARS-CoV-2) in a biological sample from a subject using methods of the present invention. This includes contacting the biological sample with a complex comprising an anti-SARS-CoV-2 antibody labeled with a first fluorophore (e.g., a donor) and an isolated labeled protein(s) with a second fluorophore, wherein the isolated labeled protein is a for example a nucleocapsid proteins (N-protein) such as a 4-P6 peptide (SEQ ID NO:34) specific to the anti-SARS-CoV-2 antibody, wherein the complex emits a fluorescence emission signal associated with fluorescence resonance energy transfer (FRET) when the first fluorophore is excited using a light source. The biological sample is incubated with the complex for a time sufficient for the anti-SARS-CoV-2 in the sample to compete for binding with the anti-SARS-CoV-2 antibody labeled with the first fluorophore; and exciting the sample using a light source to detect the fluorescence emission signal associated with FRET, wherein an absence of the fluorescence emission signal or a decrease in the fluorescence emission signal relative to the fluorescence emission signal initially emitted by the complex indicates the presence or amount of antibodies induced by SARS-CoV-2 (anti-SARS-CoV-2) in the sample.

### VII. Embodiments

Embodiment 1. A solution phase bridging assay for detecting antibodies induced by SARS-CoV-2 (anti-SARS-CoV-2) in a biological sample from a subject, the assay comprising:

contacting the sample with a first labeled protein with a donor fluorophore;

contacting the sample with a second labeled protein with an acceptor fluorophore, wherein the first and second proteins are both spike proteins (S-protein), the first and second proteins are both nucleocapsid proteins (N-proteins), or in an alternative embodiment, two S-proteins and two N-proteins;

incubating the sample for a time sufficient to generate a ternary complex of the first labeled protein with a donor fluorophore, the second labeled protein labeled with an acceptor fluorophore and the anti-SARS-CoV-2, or in the alternative embodiment, incubating the sample for a time sufficient to generate two ternary complexes, wherein (i) the first ternary complex is a S-protein labeled with a donor fluorophore, a S-protein labeled with an acceptor fluorophore and the anti-SARS-CoV-2, the (ii) second ternary complex is a N-protein labeled with a donor fluorophore, a N-protein labeled with an acceptor fluorophore, which acceptor fluorophore is optionally different than the S acceptor fluorophore and the anti-SARS-CoV-2; and

exciting the sample having the ternary complex(es) using a light source to detect a fluorescence emission signal associated with fluorescence resonance energy transfer (FRET) when the donor fluorophore is excited.

Embodiment 2. The method according to embodiment 1, wherein the antibodies are members selected from the group consisting of IgA antibodies, IgM antibodies, IgG antibodies or a combination thereof.
Embodiment 3. The method according to embodiment 1 or 2, wherein the S-protein is a member selected from the group consisting of a mammalian cell expressed recombinant spike protein of SARS-CoV-2, a fragment thereof or a synthetic S-peptide.
Embodiment 4. The method according to embodiment 1 or 2, wherein the N-protein is a member selected from the group consisting of mammalian cell expressed recombinant nucleocapsid protein of SARS-CoV-2, a fragment thereof or a synthetic N-peptide.
Embodiment 5. The method according to any one of embodiments 1 to 4, wherein the FRET emission signals are time resolved FRET emission signals.
Embodiment 6. The method according to any one of embodiments 1 to 5, wherein the sample is a biological sample.
Embodiment 7. The method according to embodiment 6, wherein the biological sample is selected from the group consisting of whole blood, urine, a fecal specimen, plasma, serum, saliva or an oral fluid.
Embodiment 8. The method according to embodiment 7, wherein the biological sample is serum.
Embodiment 9. The method according to any one of embodiments 1 to 8, wherein in the alternative embodiment, two S-proteins and two N-proteins are used to generate two ternary complexes, wherein (i) the first ternary complex is a S-protein labeled with a donor fluorophore, a S-protein labeled with an acceptor fluorophore and the anti-SARS-CoV-2, the (ii) second ternary complex is a N-protein labeled with a donor fluorophore, a N-protein labeled with a different acceptor fluorophore and the anti-SARS-CoV-2, to obtain total antibody.
Embodiment 10. The method according to any one of embodiments 1 to 9, wherein the donor fluorophore is a cryptate.
Embodiment 11. The method according to any one of embodiments 1 to 10, wherein the cryptate is a terbium cryptate.
Embodiment 12. The method according to any one of embodiments 1 to 11, wherein the acceptor fluorophore or the different acceptor fluorophore is independently selected from the group consisting of fluorescein-like (green zone) dyes, Cy5, DY-647, phycoerythrin, allophycocyanin (APC), Alexa Fluor 488, Alexa Fluor 546, and Alexa Fluor 647.
Embodiment 13. The method according to any one of embodiments 1 to 12, wherein the light source provides an excitation wavelength between about 300 nm to about 400 nm.
Embodiment 14. The method according to any one of embodiments 1 to 13, wherein the fluorescence emission signals emit emission wavelengths that are between about 450 nm to 700 nm.
Embodiment 15. The method according to any one of embodiments 1 to 14, wherein an emission signal associated with a test sample with a value equal to or greater than a cut-off value is considered to be positive.
Embodiment 16. The method according to any one of embodiments 1 to 14, wherein an emission signal associated with a test sample with a value less than a cut-off value is considered to be negative.
Embodiment 17. A competitive assay method for detecting antibodies induced by SARS-CoV-2 (anti-SARS-CoV-2) in a biological sample from a subject, the assay comprising:

contacting the sample with a complex comprising an anti-SARS-CoV-2 antibody labeled with a first fluorophore and an isolated labeled protein(s) with a second fluorophore, wherein the isolated labeled protein is a spike protein (S-protein) specific to the anti-SARS-CoV-2 antibody or a nucleocapsid proteins (N-protein) specific to the anti-SARS-CoV-2 antibody, wherein the complex emits a fluorescence emission signal associated with fluorescence resonance energy transfer (FRET) when the first fluorophore is excited using a light source;

incubating the biological sample with the complex for a time sufficient for the anti-SARS-CoV-2 in the sample to compete for binding with the anti-SARS-CoV-2 antibody labeled with the first fluorophore; and

exciting the sample using a light source to detect the fluorescence emission signal associated with FRET, wherein an absence of the fluorescence emission signal or a decrease in the fluorescence emission signal relative to the fluorescence emission signal initially emitted by the complex indicates the presence or amount of antibodies induced by SARS-CoV-2 (anti-SARS-CoV-2) in the sample.

Embodiment 18. The method according to embodiment 17, wherein the first fluorophore is a donor fluorophore.
Embodiment 19. The method according to embodiment 17, wherein the second fluorophore is an acceptor fluorophore.
Embodiment 20. The method according to any one of embodiments 17 to 19, wherein the antibodies are members selected from the group consisting of IgA antibodies, IgM antibodies, IgG antibodies or a combination thereof.
Embodiment 21. The method according to any one of embodiments 17 to 20, wherein the S-protein is a mammalian cell expressed recombinant spike protein of SARS-CoV-2 or fragment thereof.
Embodiment 22. The method according to any one of embodiments 17 to 20, wherein the N-protein is a mammalian cell expressed recombinant nucleocapsid protein of SARS-CoV-2 or fragment thereof.
Embodiment 23. The method according to any one of embodiments 17 to 22, wherein the FRET emission signals are time resolved FRET emission signals.
Embodiment 24. The method according to any one of embodiments 17 to 23, wherein the sample is a biological sample.
Embodiment 25. The method according to embodiment 24, wherein the biological sample is selected from the group consisting of whole blood, urine, a fecal specimen, plasma, and serum.
Embodiment 26. The method according to embodiment 25, wherein the biological sample is serum.
Embodiment 27. The method according to embodiment 18, wherein the donor fluorophore is a cryptate.
Embodiment 28. The method according to embodiment 27, wherein the cryptate is a terbium cryptate.
Embodiment 29. The method according to embodiment 19, wherein the acceptor fluorophore is selected from the group consisting of fluorescein-like (green zone) dyes, Cy5, DY-647, phycoerythrin, allophycocyanin (APC), Alexa Fluor 488, Alexa Fluor 546, and Alexa Fluor 647.
Embodiment 30. The method according to any one of embodiments 17 to 29, wherein the light source provides an excitation wavelength between about 300 nm to about 400 nm.
Embodiment 31. The method according to any one of embodiments 17 to 31, wherein the fluorescence emission signals emit emission wavelengths that are between about 450 nm to 700 nm.
Embodiment 32. A sandwich assay for detecting IgM antibodies induced by SARS-CoV-2 (anti-SARS-CoV-2) in a biological sample from a subject, the method comprising:
contacting a sample with a goat anti-human IgM labeled with a first fluorophore;
contacting the sample with a SARS COV-2 protein labeled with a second fluorophore; incubating the sample for a time sufficient to form a ternary complex comprising a goat anti-human IgM labeled with a first fluorophore, a SARS COV-2 protein labeled with a second fluorophore and a human IgM antibody; and
exciting the sample having the ternary complex using a light source to detect a fluorescence emission signal associated with fluorescence resonance energy transfer (FRET).
Embodiment 33. The method according to embodiment 32, wherein the first fluorophore is a donor fluorophore.
Embodiment 34. The method according to embodiment 32, wherein the second fluorophore is an acceptor fluorophore.
Embodiment 35. The method according to any one of embodiments 32 to 34, wherein SARS COV-2 protein is a S-protein, which is a member selected from the group consisting of a mammalian cell expressed recombinant spike protein of SARS-CoV-2, a fragment thereof or a synthetic S-peptide.
Embodiment 36. The method according to any one of embodiments 32 to 34, wherein the SARS COV-2 protein is a N-protein, which is a member selected from the group consisting of mammalian cell expressed recombinant nucleocapsid protein of SARS-CoV-2, a fragment thereof or a synthetic N-peptide.
Embodiment 37. A method for detecting total amount of antibody including IgG and IgM in a sample of a subject, the method comprising:

contacting a sample with (i) a first ternary complex comprising a first protein having a first fluorophore, a second protein having a second fluorophore, an anti-SARS CoV-2 IgG antibody; and (ii) a second ternary complex comprising the first protein having the first fluorophore, the second protein having the second fluorophore, an anti-SARS CoV-2 IgM antibody;

incubating the biological sample with the ternary complexes (i) and (ii) for a time sufficient for the anti-SARS-CoV-2 IgG and IgM in the sample to compete for binding for the proteins labeled with the first and the second fluorophores; and

exciting the sample using a light source to detect the fluorescence emission signal associated with FRET, wherein an absence of the fluorescence emission signal or a decrease in the fluorescence emission signal relative to the fluorescence emission signal initially emitted by the complex indicates the presence or amount of antibodies induced by SARS-CoV-2 (anti-SARS-CoV-2) in the sample.

Embodiment 38. The method according to embodiment 37, wherein the first fluorophore is a donor fluorophore.
Embodiment 39. The method according to embodiment 37, wherein the second fluorophore is an acceptor fluorophore.
Embodiment 40. The method of any one of embodiments 37-39, wherein the method further comprises adding an anti-human IgM antibody having a third fluorophore to ascertain the proportion or amount of IgM which makes up the total antibodies.
Embodiment 41. The method of any one of embodiments 37-40, wherein the first and second proteins are both spike proteins (S-protein) or wherein both are nucleocapsid proteins (N-proteins).
Embodiment 42. The method according to embodiment 41, wherein the S-protein is selected from the group consisting of a mammalian cell expressed recombinant spike protein of SARS-CoV-2, a fragment thereof or a synthetic S-peptide.
Embodiment 43. The method according to embodiment 41, wherein the N-protein is selected from the group consisting of mammalian cell expressed recombinant nucleocapsid protein of SARS-CoV-2, a fragment thereof or a synthetic N-peptide.
Embodiment 44. A multiplex inhibition assay for detecting IgG and IgM antibodies to S-protein and N-protein in a sample of a subject, the method comprising:

contacting a sample with (i) a first ternary complex comprising a S-protein having a donor fluorophore, a monoclonal anti-S-SARS CoV-2 IgG antibody having a first acceptor fluorophore attached thereto;

contacting a sample with (ii) a second ternary complex comprising a N-protein having a donor fluorophore, a monoclonal anti-N-SARS CoV-2 IgG antibody having a second acceptor fluorophore attached thereto;

incubating the biological sample with the ternary complexes (i) and (ii) for a time sufficient for the anti-SARS-CoV-2 IgG and IgM in the sample to compete for binding for the N and S labeled proteins; and

exciting the sample using a light source to detect the fluorescence emission signal associated with FRET, wherein an absence of the fluorescence emission signal or a decrease in the fluorescence emission signal relative to the fluorescence emission signal initially emitted by the complex indicates the presence or amount of IgG and IgM antibodies induced by SARS-CoV-2 (anti-SARS-CoV-2) in the sample.

Embodiment 45. The method according to embodiment 44, wherein the S-protein is selected from the group consisting of a mammalian cell expressed recombinant spike protein of SARS-CoV-2, a fragment thereof or a synthetic S-peptide.
Embodiment 46. The method according to embodiment 44, wherein the N-protein is selected from the group consisting of mammalian cell expressed recombinant nucleocapsid protein of SARS-CoV-2, a fragment thereof or a synthetic N-peptide.
Embodiment 47. A kit for the detection of antibodies induced by SARS-CoV-2 (anti-SARS-CoV-2) in a biological sample from a subject, the kit comprising:

a lyophilized first labeled protein with a donor fluorophore; and

a lyophilized second labeled protein with an acceptor fluorophore, wherein the first and second proteins are both spike proteins (S-protein) or wherein the first and second proteins are both nucleocapsid proteins (N-proteins).

Embodiment 48. The kit according to embodiment 47, wherein the S-protein is a mammalian cell expressed recombinant spike protein of SARS-CoV-2 or fragment thereof.
Embodiment 49. The kit according to embodiment 47, wherein the N-protein is a mammalian cell expressed recombinant nucleocapsid protein of SARS-CoV-2 or fragment thereof.
Embodiment 50. The kit according to embodiment 47, wherein the cryptate is a terbium cryptate.
Embodiment 51. The kit according to embodiment 47, wherein the acceptor fluorophore is selected from the group consisting of fluorescein-like (green zone) dyes, Cy5, DY-647, phycoerythrin, allophycocyanin (APC), Alexa Fluor 488, Alexa Fluor 546, and Alexa Fluor 647.
Embodiment 52. The kit according to embodiment 47, wherein the kit further comprises instructions for use.
Embodiment 53. The kit according to embodiment 47, wherein the kit further comprises a cuvette having lyophilized reagents.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

### INFORMAL SEQUENCE LISTING

SEQ ID NO: 1
ORIGIN

| | |
|---|---|
| 1 | mfvflvllpl vssqcvnltt rtqlppaytn sftrgvyypd kvfrssvlhs tqdlflpffs |
| 61 | nvtwfhaihv sgtngtkrfd npvlpfndgv yfasteksni irgwifgtti dsktqsiliv |
| 121 | nnatnvvikv cefqfcndpf igvyyhknnk swmesefrvy ssannctfey vsqpflmdle |
| 181 | gkqgnfknlr efvfknidgy fkiyskhtpi nlvrdlpqgf saleplvdlp iginitrfqt |
| 241 | llalhrsylt pgdsssgwta gaaayyvgyl qprtfllkyn engtitdavd caldplsetk |
| 301 | ctlksftvek giyqtsnfrv qptesivrfp nitnlcpfge vfnatrfasv yawnrkrisn |
| 361 | cvadysvlyn sasfstfkcy gvsptklndl cftnvyadsf virgdevrqi apgqtgkiad |
| 421 | ynyklpddft gcviawnsnn idskvggnyn ylyrlfrksn ikpferdist eiyqagstpc |
| 481 | ngvegfncyf plqsygfqpt ngvgyqpyrv vvlsfellha patvcgpkks tnlvknkcvn |
| 541 | fnfngltgtg vltesnkkfl pfqqfgrdia dttdavrdpq tleilditpc sfggvsvitp |
| 601 | gtntsnqvav lyqdvnctev pvaihadqlt ptwrvystgs nvfxtragcl igaehvnnsy |
| 661 | ecdipigagi casyqtqtns prrarsvasq siiaytmslg aensvaysnn siaiptnfti |
| 721 | svtteilpvs mtktsvdctm yicgdstecs nlllqygsfc tqlnraltgi aveqdkntqe |
| 781 | vfaqvkqiyk tppikdfggf nfsqilpdps kpskrsfied llfnkvtlad agfikqygdc |
| 841 | lgdiaardli caqkfngltv ipplltdemi aqytsallag titsgwtfga gaalqipfam |
| 901 | qmayrfngig vtqnvlyenq klianqfnsa igkiqdslss tasalgklqd vvnqnaqaln |
| 961 | tlvkqlssnf gaissvindi lsrldkveae vqidrlitgr iqslqtyvtq qliraaeira |
| 1021 | sanlaatkms ecvlgqskrv dfcgkgyhlm sfpqsaphgv vflhvtyvpa qeknfttapa |
| 1081 | ichdgkahfp regvfvsngt hwfvtqrnfy epqiittdnt fvsgncdvvi givnntvydp |
| 1141 | iqpeldsfke eldkyfknht spdvdlgdis ginasvvniq keidrineva knineslidl |
| 1201 | qelgkyeqyi kwpwyiwlgf iagliaivmv timlccmtsc csclkgccsc gscckfdedd |
| 1261 | sepvlkgvkl hyt |

//
SEQ ID NO: 2
ORIGIN

| | |
|---|---|
| 1 | rvqptesivr fpnitnlcpf gevfnatrfa svyawnrkri sncvadysvl ynsasfstfk |
| 61 | cygvsptkln dlcftnvyad sfvirgdevr qiapgqtgki adynyklpdd ftgcviawns |
| 121 | nnldskvggn ynylyrlfrk snlkpferdi steiyqagst pcngvegfnc yfplqsygfq |
| 181 | ptngvgyqpy rvvvlsfell hapatvcgpk kstnlvknkc vnfhhhhhh |

SEQ ID NO: 3
ORIGIN

| | |
|---|---|
| 1 | gvtqnvlyen qklianqfns aigkiqdsls stasalgklq dvvnqnaqal ntlvkqlssn |
| 61 | fgaissvlnd ilsrldkves ggrggpdvdl gdisginasv vniqkeidrl nevaknlnes |
| 12 | lidlqelgky gg |

//
SEQ ID NO: 4
ORIGIN

| | |
|---|---|
| 1 | msdngpqnqr napritfggp sdstgsnqng ersgarskqr rpqglpnnta swftaltqhg |
| 61 | kedlkfprgq gvpintnssp ddqigyyrra trrirggdgk mkdisprwyf yylgtgpeag |
| 121 | ipygankdgi iwvategaln tpkdhigtrn pannaaivlq lpqgttlpkg fyaegsrggs |
| 181 | qasscsssrs rnssrnstpg ssrgtsparm agnggdaala lllldrlnql eskmsgkgqq |
| 241 | qqgqtvtkks aaeaskkprq krtatkaynv tqafgrrgpe qtqgnfgdqe lirqgtdykh |
| 301 | wpqiaqfaps asaffgmsri gmevtpsgtw ltytgaikld dkdpnfkdqv illnkhiday |
| 361 | ktfpptepkk dkkkkadetq alpqrqkkqq tvtilpaadl ddfskqlqqs mssadstqa |

//

What is claimed is:

1. A solution phase bridging assay for detecting antibodies induced by SARS-CoV-2 (anti-SARS-CoV-2) in a biological sample from a subject, the assay comprising:
contacting the sample with a first labeled protein with a donor fluorophore;
contacting the sample with a second labeled protein with an acceptor fluorophore, wherein the first and second proteins are both spike proteins (S-protein), the first and second proteins are both nucleocapsid proteins (N-proteins), or in an alternative embodiment, two S-proteins and two N-proteins;
incubating the sample for a time sufficient to generate a ternary complex of the first labeled protein with a donor fluorophore, the second labeled protein labeled with an acceptor fluorophore and the anti-SARS-CoV-2, or in the alternative embodiment, incubating the sample for a time sufficient to generate two ternary complexes, wherein (i) the first ternary complex is a S-protein labeled with a donor fluorophore, a S-protein labeled with an acceptor fluorophore and the anti-SARS-CoV-2, the (ii) second ternary complex is a N-protein labeled with a donor fluorophore, a N-protein labeled with an acceptor fluorophore, which acceptor fluorophore is optionally different than the S acceptor fluorophore and the anti-SARS-CoV-2; and
exciting the sample having the ternary complex(es) using a light source to detect a fluorescence emission signal associated with fluorescence resonance energy transfer (FRET) when the donor fluorophore is excited.

2. The method according to claim 1, wherein the antibodies are members selected from the group consisting of IgA antibodies, IgM antibodies, IgG antibodies or a combination thereof.

3. The method according to claim 1, wherein the S-protein is a member selected from the group consisting of a mammalian cell expressed recombinant spike protein of SARS-CoV-2, a fragment thereof or a synthetic S-peptide.

4. The method according to claim 1, wherein the N-protein is a member selected from the group consisting of mammalian cell expressed recombinant nucleocapsid protein of SARS-CoV-2, a fragment thereof or a synthetic N-peptide.

5. The method according to claim 1, wherein the FRET emission signals are time resolved FRET emission signals.

6. The method according to claim 1, wherein the sample is a biological sample.

7. The method according to claim 6, wherein the biological sample is selected from the group consisting of whole blood, urine, a fecal specimen, plasma, serum, saliva or an oral fluid.

8. The method according to claim 7, wherein the biological sample is serum.

9. The method according to claim 1, wherein in the alternative embodiment, two S-proteins and two N-proteins are used to generate two ternary complexes, wherein (i) the first ternary complex is a S-protein labeled with a donor fluorophore, a S-protein labeled with an acceptor fluorophore and the anti-SARS-CoV-2, the (ii) second ternary complex is a N-protein labeled with a donor fluorophore, a N-protein labeled with a different acceptor fluorophore and the anti-SARS-CoV-2, to obtain total antibody.

10. The method according to claim 1, wherein the donor fluorophore is a cryptate.

11. The method according to claim 1, wherein the cryptate is a terbium cryptate.

12. The method according to claim 1, wherein the acceptor fluorophore or the different acceptor fluorophore is independently selected from the group consisting of fluorescein-like (green zone) dyes, Cy5, DY-647, phycoerythrin, allophycocyanin (APC), Alexa Fluor 488, Alexa Fluor 546, and Alexa Fluor 647.

13. The method according to claim 1, wherein the light source provides an excitation wavelength between about 300 nm to about 400 nm.

14. The method according to claim 1, wherein the fluorescence emission signals emit emission wavelengths that are between about 450 nm to 700 nm.

15. The method according to claim 1, wherein an emission signal associated with a test sample with a value equal to or greater than a cut-off value is considered to be positive.

16. The method according to claim 1, wherein an emission signal associated with a test sample with a value less than a cut-off value is considered to be negative.

17. A competitive assay method for detecting antibodies induced by SARS-CoV-2 (anti-SARS-CoV-2) in a biological sample from a subject, the assay comprising:
contacting the sample with a complex comprising an anti-SARS-CoV-2 antibody labeled with a first fluorophore and an isolated labeled protein(s) with a second fluorophore, wherein the isolated labeled protein is a spike protein (S-protein) specific to the anti-SARS-CoV-2 antibody or a nucleocapsid proteins (N-protein) specific to the anti-SARS-CoV-2 antibody, wherein the complex emits a fluorescence emission signal associated with fluorescence resonance energy transfer (FRET) when the first fluorophore is excited using a light source;
incubating the biological sample with the complex for a time sufficient for the anti-SARS-CoV-2 in the sample to compete for binding with the anti-SARS-CoV-2 antibody labeled with the first fluorophore; and
exciting the sample using a light source to detect the fluorescence emission signal associated with FRET, wherein an absence of the fluorescence emission signal or a decrease in the fluorescence emission signal relative to the fluorescence emission signal initially emitted by the complex indicates the presence or amount of antibodies induced by SARS-CoV-2 (anti-SARS-CoV-2) in the sample.

18. The method according to claim 17, wherein the first fluorophore is a donor fluorophore.

19. The method according to claim 17, wherein the second fluorophore is an acceptor fluorophore.

20. The method according to claim 17, wherein the antibodies are members selected from the group consisting of IgA antibodies, IgM antibodies, IgG antibodies or a combination thereof.


SEQUENCE LISTING (50 sequences)

SEQ ID NO: 1 - protein, 1273 aa, Severe acute respiratory syndrome coronavirus 2
  MOD_RES 644: note = Any amino acid
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFXTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 229 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH

SEQ ID NO: 3 - protein, 132 aa, Severe acute respiratory syndrome coronavirus 2
GVTQNVLYENQKLIANQFNSAIGKIQDSLSSTASALGKLQDVVNQNAQALNTLVKQLSSNFGAISSVLNDILSRLDKVESGGRGGPDVDLGDISGINASVVNIQKEIDRLNEVAKNLNESLIDLQELGKYGG

SEQ ID NO: 4 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSCSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 5 - protein, 37 aa, synthetic construct
  REGION 1..37: note = source = /note="Description of Artificial Sequence: Synthetic polypeptide"
KGIYQTSNFRVQPTESIVRFPNITNLCPFGEVFNATK

SEQ ID NO: 6 - protein, 31 aa, synthetic construct
  REGION 1..31: note = source = /note="Description of Artificial Sequence: Synthetic polypeptide"
GAALQIPFAMQMAYRFNGIGVTQNVLYENQK

SEQ ID NO: 7 - protein, 36 aa, synthetic construct
  REGION 1..36: note = source = /note="Description of Artificial Sequence: Synthetic polypeptide"
AISSVLNDILSRLDKVEAEVQIDRLITGRLQSLQTK

SEQ ID NO: 8 - protein, 36 aa, synthetic construct
  REGION 1..36: note = source = /note="Description of Artificial Sequence: Synthetic polypeptide"
YVTQQLIRAAEIRASANLAATKMSECVLGQSKRVDK

SEQ ID NO: 9 - protein, 21 aa, synthetic construct
  REGION 1..21: note = source = /note="Description of Artificial Sequence: Synthetic peptide"
TSSMRGVYYPDEIFRSDTLYL

SEQ ID NO: 10 - protein, 20 aa, synthetic construct
  REGION 1..20: note = source = /note="Description of Artificial Sequence: Synthetic peptide"
KSNVVRGWVFGSTMNNKSQS

SEQ ID NO: 11 - protein, 20 aa, synthetic construct
  REGION 1..20: note = source = /note="Description of Artificial Sequence: Synthetic peptide"
YKGYQPIDVVRDLPSGFNTL

SEQ ID NO: 12 - protein, 21 aa, synthetic construct
  REGION 1..21: note = source = /note="Description of Artificial Sequence: Synthetic peptide"
TDAVDCSQNPLAELKCSVKSF

SEQ ID NO: 13 - protein, 20 aa, synthetic construct
  REGION 1..20: note = source = /note="Description of Artificial Sequence: Synthetic peptide"
KGIYQTSNFRVVPSGDVVRF

SEQ ID NO: 14 - protein, 20 aa, synthetic construct
  REGION 1..20: note = source = /note="Description of Artificial Sequence: Synthetic peptide"
TKFPSVYAWERKKISNCVAD

SEQ ID NO: 15 - protein, 20 aa, synthetic construct
  REGION 1..20: note = source = /note="Description of Artificial Sequence: Synthetic peptide"
YNYKYRYLRHGKLRPFERDI

SEQ ID NO: 16 - protein, 20 aa, synthetic construct
  REGION 1..20: note = source = /note="Description of Artificial Sequence: Synthetic peptide"
PSSKRFQPFQQFGRDVSDFT

SEQ ID NO: 17 - protein, 23 aa, synthetic construct
  REGION 1..23: note = source = /note="Description of Artificial Sequence: Synthetic peptide"
CANLLLQYGSFCTQLNRALSGIA

SEQ ID NO: 18 - protein, 23 aa, synthetic construct
  REGION 1..23: note = source = /note="Description of Artificial Sequence: Synthetic peptide"
RNTREVFAQVKQMYKTPTLKYFG

SEQ ID NO: 19 - protein, 20 aa, synthetic construct
  VARIANT 8: note = /replace="Lys"
  VARIANT 20: note = /replace="Leu"
  SITE 1..20: note = /note="Variant residues given in the sequence have no preference with respect to those in the annotations for variant positions"
  REGION 1..20: note = source = /note="Description of Artificial Sequence: Synthetic peptide"
FKLPLGINITNFRAILTAFS

SEQ ID NO: 20 - protein, 20 aa, synthetic construct
  VARIANT 3: note = /replace="Lys"
  SITE 1..20: note = /note="Variant residues given in the sequence have no preference with respect to those in the annotations for variant positions"
  REGION 1..20: note = source = /note="Description of Artificial Sequence: Synthetic peptide"
PTTFMLKYDENGTITDAVDC

SEQ ID NO: 21 - protein, 20 aa, synthetic construct
  VARIANT 7: note = /replace="Ser"
  SITE 1..20: note = /note="Variant residues given in the sequence have no preference with respect to those in the annotations for variant positions"
  REGION 1..20: note = source = /note="Description of Artificial Sequence: Synthetic peptide"
VLYNSTFFSTFKCYGVSATK

SEQ ID NO: 22 - protein, 20 aa, synthetic construct
  VARIANT 10: note = /replace="Lys"
  SITE 1..20: note = /note="Variant residues given in the sequence have no preference with respect to those in the annotations for variant positions"
  REGION 1..20: note = source = /note="Description of Artificial Sequence: Synthetic peptide"
PALNCYWPLNDYGFYTTSGI

SEQ ID NO: 23 - protein, 20 aa, synthetic construct
  VARIANT 6: note = /replace="Ile"
  SITE 1..20: note = /note="Variant residues given in the sequence have no preference with respect to those in the annotations for variant positions"
  REGION 1..20: note = source = /note="Description of Artificial Sequence: Synthetic peptide"
RDVSDFTDSVRDPKTSEILD

SEQ ID NO: 24 - protein, 20 aa, synthetic construct
  VARIANT 10: note = /replace="Pro"
  SITE 1..20: note = /note="Variant residues given in the sequence have no preference with respect to those in the annotations for variant positions"
  REGION 1..20: note = source = /note="Description of Artificial Sequence: Synthetic peptide"
YQDVNCTDVSTAIHADQLTP

SEQ ID NO: 25 - protein, 20 aa, synthetic construct
  VARIANT 12: note = /replace="Leu"
  SITE 1..20: note = /note="Variant residues given in the sequence have no preference with respect to those in the annotations for variant positions"
  REGION 1..20: note = source = /note="Description of Artificial Sequence: Synthetic peptide"
SNNTIAIPTNFSISITTEVM

SEQ ID NO: 26 - protein, 20 aa, synthetic construct
  VARIANT 7: note = /replace="Ala"
  VARIANT 18: note = /replace="Val"
  SITE 1..20: note = /note="Variant residues given in the sequence have no preference with respect to those in the annotations for variant positions"
  REGION 1..20: note = source = /note="Description of Artificial Sequence: Synthetic peptide"
QYGSFCTQLNRALSGIAAEQ

SEQ ID NO: 27 - protein, 20 aa, synthetic construct
  VARIANT 5: note = /replace="Ala"
  SITE 1..20: note = /note="Variant residues given in the sequence have no preference with respect to those in the annotations for variant positions"
  REGION 1..20: note = source = /note="Description of Artificial Sequence: Synthetic peptide"
GIGVTQNVLYENQKQIANQF

SEQ ID NO: 28 - protein, 20 aa, synthetic construct
  VARIANT 3: note = /replace="Glu"
  SITE 1..20: note = /note="Variant residues given in the sequence have no preference with respect to those in the annotations for variant positions"
  REGION 1..20: note = source = /note="Description of Artificial Sequence: Synthetic peptide"
IQKEIDRLNEVAKNLNESLI

SEQ ID NO: 29 - protein, 27 aa, synthetic construct
  REGION 1..27: note = source = /note="Description of Artificial Sequence: Synthetic peptide"
GRRPQGLPNNTASWFTALTQHGKEDLK

SEQ ID NO: 30 - protein, 33 aa, synthetic construct
  REGION 1..33: note = source = /note="Description of Artificial Sequence: Synthetic polypeptide"
QLPQGTTLPKGFYAEGSRGGSQASSRSSSRSRK

SEQ ID NO: 31 - protein, 48 aa, synthetic construct
  REGION 1..48: note = source = /note="Description of Artificial Sequence: Synthetic polypeptide"
QQQGQTVTKKSAAEASKKPRQKRTATKAYNVTQAFGRRGPEQTQGNFK

SEQ ID NO: 32 - protein, 40 aa, synthetic construct
  REGION 1..40: note = source = /note="Description of Artificial Sequence: Synthetic polypeptide"
IRQGTDYKHWPQIAQFAPSASAFFGMSRIGMEVTPSGTWK

SEQ ID NO: 33 - protein, 28 aa, synthetic construct
  REGION 1..28: note = source = /note="Description of Artificial Sequence: Synthetic peptide"
LLNKHIDAYKTEPKTFPPKDKKKKADEK

SEQ ID NO: 34 - protein, 27 aa, synthetic construct
  REGION 1..27: note = source = /note="Description of Artificial Sequence: Synthetic peptide"
TQALPQRQKKQQTVTLLPAADLDDFSK

SEQ ID NO: 35 - protein, 26 aa, synthetic construct
  REGION 1..26: note = source = /note="Description of Artificial Sequence: Synthetic peptide"
PSDSTGSNQNGERSGARSKQGRRPQK

SEQ ID NO: 36 - protein, 28 aa, synthetic construct
  REGION 1..28: note = source = /note="Description of Artificial Sequence: Synthetic peptide"
LLNKHIDAYKTEPKTFPPKDKKKKADEK

SEQ ID NO: 37 - protein, 23 aa, synthetic construct
  REGION 1..23: note = source = /note="Description of Artificial Sequence: Synthetic peptide"
MSDNGPQSNQRSAPRITFGGPTD

SEQ ID NO: 38 - protein, 22 aa, synthetic construct
  REGION 1..22: note = source = /note="Description of Artificial Sequence: Synthetic peptide"
PTDSTDNNQNGGRNGARPKQRR

SEQ ID NO: 39 - protein, 22 aa, synthetic construct
  REGION 1..22: note = source = /note="Description of Artificial Sequence: Synthetic peptide"
GARPKQRRPQGLPNNTASWFTA

SEQ ID NO: 40 - protein, 22 aa, synthetic construct
  REGION 1..22: note = source = /note="Description of Artificial Sequence: Synthetic peptide"
QLPQGTTLPKGFYAEGSRGGSQ

SEQ ID NO: 41 - protein, 22 aa, synthetic construct
  REGION 1..22: note = source = /note="Description of Artificial Sequence: Synthetic peptide"
DGKMKELSPRWYFYYLGTGPEA

SEQ ID NO: 42 - protein, 22 aa, synthetic construct
  REGION 1..22: note = source = /note="Description of Artificial Sequence: Synthetic peptide"
SRGGSQASSRSSSRSRGNSRNS

SEQ ID NO: 43 - protein, 22 aa, synthetic construct
  REGION 1..22: note = source = /note="Description of Artificial Sequence: Synthetic peptide"
RNSTPGSSRGNSPARMASGGGE

SEQ ID NO: 44 - protein, 25 aa, synthetic construct
  REGION 1..25: note = source = /note="Description of Artificial Sequence: Synthetic peptide"
GGETALALLLLDRLNQLESKVSGKG

SEQ ID NO: 45 - protein, 24 aa, synthetic construct
  REGION 1..24: note = source = /note="Description of Artificial Sequence: Synthetic peptide"
QTVTKKSAAEASKKPRQKRTATKQ

SEQ ID NO: 46 - protein, 22 aa, synthetic construct
  REGION 1..22: note = source = /note="Description of Artificial Sequence: Synthetic peptide"
KPRQKRTATKQYNVTQAFGRRG

SEQ ID NO: 47 - protein, 22 aa, synthetic construct
  REGION 1..22: note = source = /note="Description of Artificial Sequence: Synthetic peptide"
NKHIDAYKTFPPTEPKKDKKKK

SEQ ID NO: 48 - protein, 20 aa, synthetic construct
  REGION 1..20: note = source = /note="Description of Artificial Sequence: Synthetic peptide"
KDKKKKTDEAQPLPQRQKKQ

SEQ ID NO: 49 - protein, 23 aa, synthetic construct
  REGION 1..23: note = source = /note="Description of Artificial Sequence: Synthetic peptide"
QRQKKQPTVTLLPAADMDDFSRQ

SEQ ID NO: 50 - protein, 22 aa, synthetic construct
  REGION 1..22: note = source = /note="Description of Artificial Sequence: Synthetic peptide"
MDDFSRQLQNSMSGASADSTQA